(19) United States
(12) Patent Application Publication
(10) Pub. No.: US 2011/0301697 A1
(43) Pub. Date: Dec. 8, 2011

(54) MANUFACTURE, METHOD AND USE OF DRUG-ELUTING MEDICAL DEVICES FOR PERMANENTLY KEEPING BLOOD VESSELS OPEN

(75) Inventors: Erika Hoffmann, Eschweiler (DE); Michael Hoffmann, Eschweiler (DE); Roland Horres, Stolberg (DE); Sabine Küsters, Niederzier (DE)

(73) Assignee: Hemoteq AG

(21) Appl. No.: 13/161,161

(22) Filed: Jun. 15, 2011

Related U.S. Application Data
(63) Continuation of application No. 12/307,261, filed on Apr. 10, 2009.

(51) Int. Cl. A61F 2/82; A61P 37/06; A61P 35/00; A61K 31/436

(52) U.S. Cl. 623/1.46; 424/400; 424/278.1

(57) ABSTRACT

The invention relates to stents and catheter balloons having optimized coatings for eluting rapamycin as well as methods for manufacturing these coatings.

### PRIORITY CLAIM

This application is a continuation application of U.S. patent application Ser. No. 12/521,863, entitled “MANUFACTURE, METHOD AND USE OF DRUG-ELUTING MEDICAL DEVICES FOR PERMANENTLY KEEPING BLOOD VESSELS OPEN” which was filed on Apr. 10, 2009.

### BACKGROUND OF THE INVENTION

1. Field of the Invention

The invention relates to stents and catheter balloons having at least one layer which contains at least one antiproliferative, immunosuppressive, anti-inflammatory, antimycotic and/or antithrombotic active agent, methods of manufacturing these medical devices as well as their use for preventing restenosis.

2. Description of the Relevant Art

In the human body the blood gets only in cases of injuries in contact with surfaces other than the inside of natural blood vessels. Consequently, the blood coagulation system gets always activated to reduce the bleeding and to prevent a life-threatening loss of blood if blood gets in contact with foreign surfaces. Due to the fact that an implant also represents a foreign surface all patients, who receive an implant which is in permanent contact with blood, are treated for the duration of the blood contact with drugs, so called anticoagulants which suppress the blood coagulation, wherein sometimes considerable side effects have to be taken in account. The described risk of thrombosis occurs also as one of the risk factors in the utilization of vessel supports, so called stents, in blood-containing vessels. The stent serves for permanent expansion of the vessel walls in the occurrence of vessel narrowings and occlusions, e.g. by arteriosclerotic changes especially of the coronary arteries. The material which is used for the stent is usually medical stainless steel, Ni—Ti alloys or Co—Cr alloys while polymeric stents are still in the phase of development. Stent thrombosis occurs in less than one percent of the cases already in the cardio catheter laboratory as early thrombosis or in two to five percent of the cases during the hospital recreation. In about five percent of the cases vessel injuries due to the intervention are caused because of the arterial locks and the possibility of causing pseudo-aneurysms by the expansion of vessels exists, too.

Likewise, in the event of a PTCA the blood coagulation gets activated by introducing a foreign body. As in this case a short term implant is concerned the problems are found more substantially in the force of the vessel dilatation which is necessary to expand or to eliminate a vessel narrowing or occlusion. An additional and very often occurring complication is restenosis, the reocclusion of the vessel. Although stents reduce the risk of a reoccurring occlusion of the vessel, they are until the present day not capable of completely preventing such restenoses or are themselves the reason for neointimal hyperplasias. In the event of especially severe cases the rate of reocclusion (restenosis) after implantation of a stent is with up to 30% one of the main reasons of a repeated hospital visit for the patients. As the rate of reocclusion after PTCA is substantially higher than compared to a stent a stent is usually implanted into patients having massive stenosis or restenosis.

An exact description of the term of restenosis cannot be found in the technical literature. The most frequently used morphologic definition of restenosis is the one which defines restenosis as a reduction of the vessel diameter to less than 50% of the normal diameter after successful PTA (percutaneous transluminal angioplasty). This is an empirically determined value the hemodynamic relevance and relation to clinical pathology of which lacks of a stable scientific foundation. In practice, the clinical aggravation of a patient is often considered as a sign of a restenosis of the formerly treated vessel segment. The vessel injuries caused during the implantation of the stent or in the event of over-dilating the vessel result in inflammation reactions which play an important role for the recovery process during the first seven days. The concurrent processes herein are among others connected with the release of growth factors which initiates an increased proliferation of the smooth muscle cells and results with this in a rapid restenosis, a renewed occlusion of the vessel, because of uncontrolled growth.

Even after a couple of weeks, when the stent is grown into the tissue of the blood vessel and totally surrounded by smooth muscle cells, cicatrisations can be too distinctive (neointimal hyperplasia) and result not only in a covering of the stent surface but in the occlusion of the total interior space of the stent.

It was tried vainly to solve the problem of restenosis by developing balloon catheters which release heparin through micro-pores and later by the coating of the stents with heparin (J. Whorle et al., European Heart Journal (2001) 22, 1808-1816). However, heparin addresses as anticoagulant only the first mentioned cause and is moreover able to unfold its total effect only in solution. This first problem is meanwhile almost totally avoidable medicamentously by application of anticoagulants. The further problem is intended to be solved now by inhibiting the growth of the smooth muscle cells locally.

This is carried out by e.g. radioactive stents or stents which contain pharmaceutical active agents the action of which is preferably antiproliferative. Originating from chemotherapy the active agent paclitaxel which prevents the division of a cell in the mitosis process by irreversible binding to the forming spindle apparatus has proven itself as successful. The cell remains in this transition state which cannot be maintained and the cell dies. However, the existing research with the paclitaxel-eluting stent shows that contrary to the same uncoated stent paclitaxel results in an increased thrombosis rate in the long-term consequence. This is based on paclitaxel's mechanism of action. The irreversible binding and stabilizing of tubulin during cell division results in that the cell is not capable of realizing other cell-maintaining functions. Finally, the cell dies. By this way the process of wound healing shall be controlled better, however, by the generation of cell material which is not viable anymore an increased inflammatory reaction and thus a stronger immunologic response is undesirably achieved. It is very difficult to comply with the dosing of paclitaxel. One the one hand the inevitable reactions which induce the process of wound healing have to be combated besides the inflammatory process which is additionally induced by paclitaxel and on the other hand the dosage must not be so small that an effect is hardly achieved. This tightrope walk often results in that even after a half year the desired endothelial layer is not formed on the stent. Either the stent struts are still uncovered and result in an increased risk that even after months the patient dies because of a thrombosis (late acute thrombosis) or the cell tissue which surrounds the stent consists of smooth muscle cells, monocytes etc. which after some time can result in an occlusion again.

As a very prosperous active agent for the same purpose of restenosis prophylaxis rapamycin (syn. sirolimus) a hydrophilic macrolid antibiotic appears. This active agent is especially utilized in transplantation medicine as immunosuppressive, wherein contrary to other immunosuppressive active agents rapamycin also inhibits tumour formation. As after a transplantation an increased risk of tumour formation exists for the patient the administration of rapamycin is advantageous because other immunosuppressives such as cyclosporin A can even promote tumour formation as is known.

Rapamycin's mechanism of action is not yet known in detail but it is attributed especially to the complex formation with the protein mTOR (mammalian target of rapamycin) a phosphatidylinositol-3 kinase of 282 kD. As mTOR is responsible for a series of cytokin-mediated signal transduction paths i.a. also for signal paths which are necessary for cell division besides the immunosuppressive effect it has also antiphlogistic, antiproliferative and even antimycotic properties.

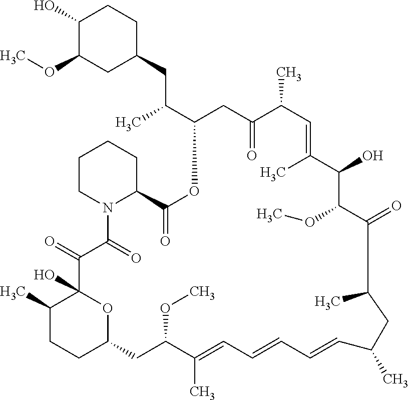

IUPAC name:

[3S-[3R*[E(1S*,3S*,4S*)],4S*,5R*,8S*,9E,12R*,14R*,15S*,16R*,18S*,19S*,26aR*]]-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]-oxaazacyclotricosine-1,7,20,21(4H,23H)-tetron monohydrate.

Proliferation is interrupted in the late G1 phase by stopping the ribosomal protein synthesis. Compared to other antiproliferative active agents rapamycin's mechanism of action can be pointed out as special likewise paclitaxel but which is strongly hydrophobic. Moreover, the immunosuppressive and antiphlogistic effects as described above are more than advantageous because also the extent of inflammatory reactions and of the total immune response as their premature control after stent implantation is decisive for the further success.

Thus, rapamycin has all of the necessary conditions for the utilization against stenoses and restenoses. Rapamycin's limited shelf life on or in an implant is to be mentioned as an additional advantage in comparison to paclitaxel because necessarily the active agent has to be effective in the first decisive weeks after stent implantation. Consequently, the endothelial cell layer which is important for the completion of a healthy healing process can completely grow over the stent and integrate it into the vessel wall.

The same mechanism of action can be found for the known derivatives of rapamycin (biolimus, everolimus) as the modification is on the molecule's functional groups which are irrelevant for the binding region of mTOR. In different clinical studies (RAVEL, SIRIUS, SIROCCO) rapamycin has shown—contrary to other active agents such as dexamethason, tacrolimus, batimastat—that in comparison to the strongly hydrophobic paclitaxel despite of different physical properties it is more than suitable for combating restenosis.

The active agent itself is no warrant for an optimal prophylaxis of restenosis. The drug-eluting stent has to meet the requirements in its entirety. Besides the determination of dosing the drug-elution has to be delayed temporally and controlled in dependence of the concentration. The drug-elution as well as the rate of drug-elution do not depend only on the physical and chemical properties of the active agent but depend also on the properties of the utilized polymer and the interactions of polymer and active agent. Stent material, stent properties and stent design are further factors which have to be considered for an optimally effective medical device.

As divisional application of EP 0950386 B1 which describes a stent with channels in the struts in which rapamycin is present under a diffusion-controlling polymer layer in EP 1407726 A1 (priority 1998) a stent is described which elutes rapamycin of a polymer matrix which is commercially available since 2002 (Cypher™ stent). There, a stent coated with parylen C is coated with a mixture of the two biostable polymers polyethylene vinylacetate (PEVA) and poly-n-butylmethylmethacrylate (PBMA) and rapamycin and provided with a diffusion-controlling drug-free topcoat of PBMA. The results with this stent have shown that allergic reactions and inflammations as well as late thromboses result in significant problems (Prof. Renu Virmani, 2002-ff). Moreover, PBMA as topcoat is problematic as it breaks during expansion and thus an uncontrolled elution of rapamycin occurs (see FIG. 1). Therewith, a general problem in the use of rapamycin appears. The controlled bioavailability of rapamycin is difficult to maintain: rapamycin as hydrophilic molecule rapidly dissolves. If the diffusion-controlling topcoat breaks the elution of rapamycin is rapid, uncontrolled and untargeted. Additionally, due to the unsatisfying elasticity of PBMA there exists the risk of delaminating larger polymer pieces which can protractedly result in further problems due to their biostability in the blood circuit (see FIG. 2).

EP 0568310 B1 claims the active agent combination of heparin and rapamycin for hyperproliferative vascular diseases. There, the description merely mentions in brief that the administration of this active agent combination can be done by means of a rapamycin-impregnated vascular stent. Examples do not exist such that only a note is concerned and therefore many questions arise. As this patent is of the year 1992 but until now only the above mentioned Cypher™ stent from Cordis Corp. based on EP 1407726 A1 is commercially available, obviously the commercial realization of a rapamycin-heparin-impregnated stent was not the primary aim of this patent.

EP 0 551 182 B1 describes and claims already with mentioning a stent a rapamycin-impregnated medicament which shall reduce or prevent mechanically induced hyperproliferative diseases. There, the rapamycin-impregnated stent is mentioned as auxiliary means for introducing rapamycin into the vessel but it is not discussed in detail. A stent impregnated with rapamycin means a pure active agent layer on the stent framework without the presence of a carrier. Technically this embodiment cannot be reasonably realized as rapamycin rapidly hydrolizes on air and easily decomposes by cleavage of the lactone bond especially in the presence of water. In addition, a pure active agent layer of rapamycin is dissolved too easily in the blood flow during the insertion of a rapamycin-coated catheter balloon or of a balloon having a rapamycin-coated stent such that it cannot be guaranteed if a sufficient amount of rapamycin on the medical device (stent or catheter balloon) is still present at the target site. Further, a pure active agent layer has the disadvantage that during dilatation the active agent is completely eluted within a short period of time because a drug-eluting coating in form of a drug-release-system is absent and thus a spontaneous elution occurs and it is not possible to take advantage of elution kinetics.

Thus, the present invention does not relate to providing rapamycin-coated stents or catheter balloons or to the use of rapamycin for the prophylaxis or treatment of restenosis, what is already state of the art, but it relates to an optimized carrier system for the delicate active agent rapamycin.

However, as already mentioned above not any active agent can be used in any way as prophylaxis of restenosis. For a successful use and long term safety of the patient independently of the quality of the uncoated implant a plurality of further conditions has to be met. The physical and chemical properties of a suitable active agent, the solvent and the optionally used matrix have to be considered as well as the interactions of these factors with each other. Only by the proper combination of these parameters the time- and dosis-controlled availability of the therapeutic is optimally regulated, wherein finally the safety and health of the patient are warranted.

### SUMMARY OF THE INVENTION

It is the object of the present invention to provide rapamycin-eluting stents and balloon catheters which guarantee a controlled and healthy healing process and permit the regeneration of a vessel wall having a complete endothelial cell layer without the above mentioned disadvantages. Thus, the object of the present invention is to provide optimized carrier systems for rapamycin which can be applied to stents, i.e. vessel supports, or catheter balloons as well as simultaneously to a crimped stent and catheter balloon, guarantee a sufficient adhesion stability and decomposition stability of the active agent rapamycin and have an elution kinetics which is suitable in the best way for prophylaxis and treatment of restenosis.

The suppression of the cellular reactions in the first days and weeks after implantation is preferably achieved by means of the antiproliferatively, immunosuppressively and antiphlogistically effective rapamycin, its equally effective derivatives/analogues and/or metabolites. Further active agents and/or active agent combinations which promote in a reasonable way the wound healing or the process of wound healing can be added.

This objective is solved by the technical teaching of the independent claims of the present invention. Further advantageous designs of the invention result from the dependent claims, the description, as well as the examples.

### DESCRIPTION OF THE FIGURES

FIG. 1: Cypher™ drug-eluting stent with 500× magnification (scanning electron microscopy). The multiple and deep cracks in the coating can be seen clearly. This results in an uncontrolled elution of active agent.

FIG. 2: Cypher™ drug-eluting stent (Cypher stent) (scanning electron microscopy); the blistering chips of the biostable polymer coating can be seen clearly. The following problems are connected therewith:
a) polymer chips which cannot be degraded by the organism are brought into the blood circulation
b) the active agent is not eluted in a targeted, controlled and properly dosed way
c) the stent's surface is exposed as a foreign surface such that the thrombosis risk is increased.

FIG. 3: Scanning electron microscopy image of a polymer-coated rapamycin-eluting stent according to this invention. The difference to the Cypher stent can be seen clearly: no cracks and no blistering of polymer chips. In the shown example a biodegradable polymer was used.

FIG. 4: Elution profile of rapamycin in the biodegradable polymer PLGA. It can be seen well that after about 400-500 hours after the “first release” (directly after implantation) a new increase in the elution rate of rapamycin occurs which we call “late burst”.

FIG. 5: Elution behavior of rapamycin from a biostable matrix.

FIG. 6: Scheme of the method of action of a pore-forming system and rapamycin-release through elution via channels and swelling The hydrophilic active agent arrives through the channels formed by the pore forming agents directly at the vessel wall. If highly swellable substances are admixed with rapamycin in a non or clearly less swellable matrix, then the active agent is pressed to the surface by the pressure generated in the swelling process (“injection model”).

FIG. 7: The matrix consists of a biostable matrix which contains a high content of pore forming agents or micro-channels through which rapamycin arrives rapidly, controlled and in high dosage to the target site. Also in this case a blistering of polymer chips or any other deficiencies are not detected.

FIG. 8: Scheme for coating rapamycin-eluting stents with matrices which form micro-channels through which rapamycin arrives at the surface. The hydrophilic active agent arrives through the channels formed by the pore forming agents directly at the vessel wall. If highly swellable substances are admixed with rapamycin in a non or clearly less swellable matrix, then the active agent is pressed to the surface by the pressure generated in the swelling process (“injection model”).

FIG. 9: An expanded balloon catheter which is completely coated with rapamycin and isopropylmyristate as adjuvant according to the invention in a combined coating method. It can be seen that even after expansion the coating is not blistering or cracking.

FIG. 10: Elution behavior of rapamycin from the Cypher stent (yellow) in comparison to a stent having a pure rapamycin layer and a topcoat of PVA (blue). The substantially accelerated elution behavior of the rapamycin/PVA-system can be clearly distinguished from Cypher.

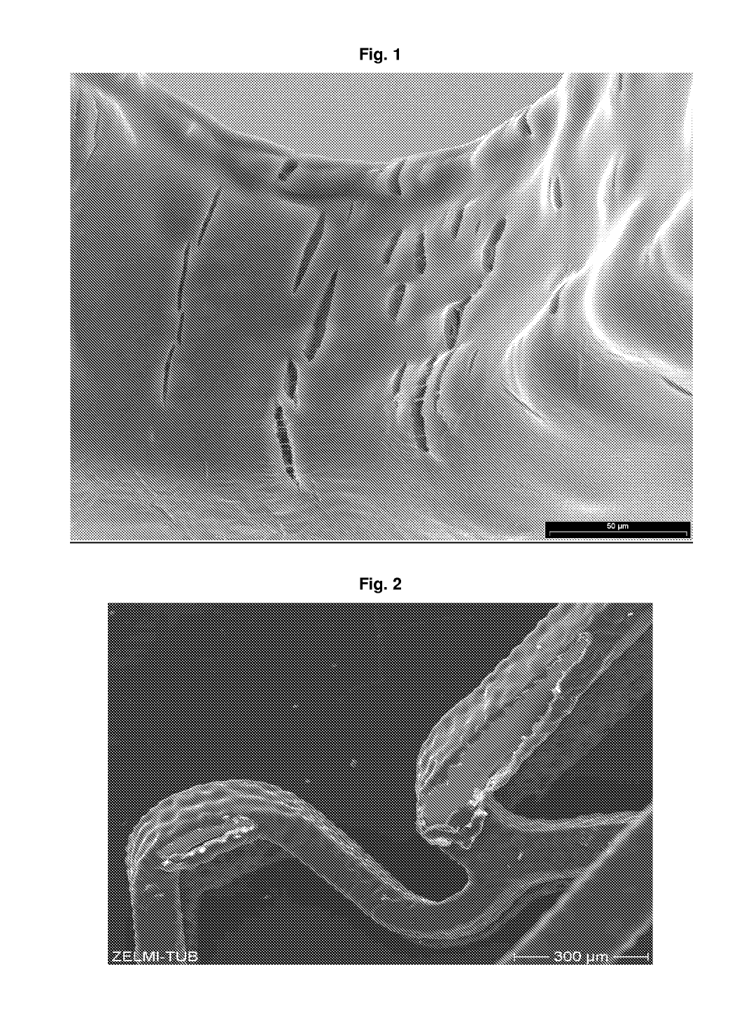

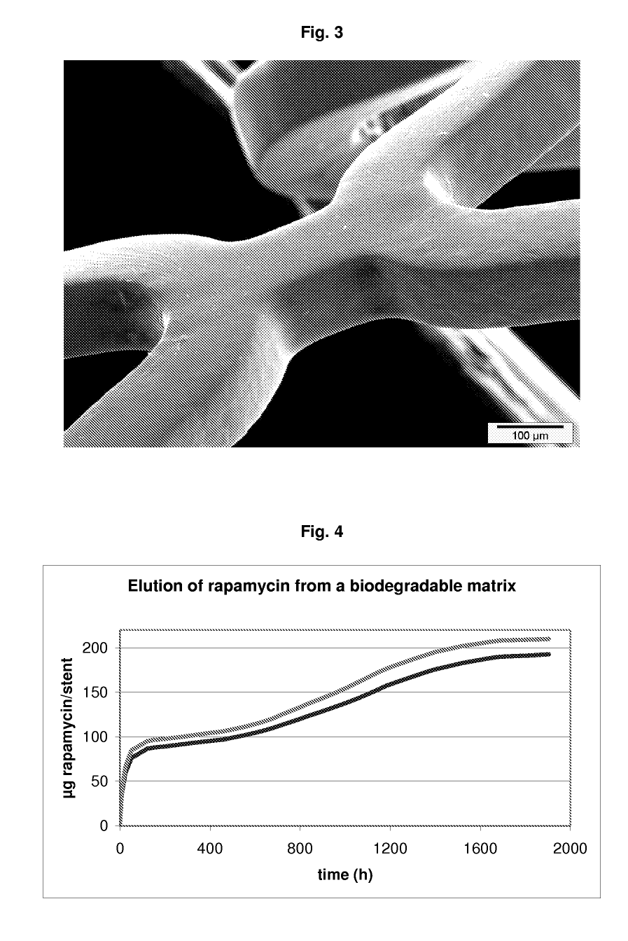

### DETAILED DESCRIPTION

The stents according to the invention have one, two or more layers, wherein at least one layer is containing rapamycin or an effective combination of rapamycin with other active agents which are complementarily and/or synergistically effective with rapamycin or is applied without a polymer carrier. Rapamycin or an active agent combination with rapamycin is bound covalently and/or adhesively to the subjacent layer or the stent surface and/or incorporated covalently and/or adhesively into the layer such that the active agent is released continuously and in small dosages and that the ongrowth of the stent surface with cells is not prevented, but an overgrowth. The combination of both effects confers to the stent according to the invention the ability of rapidly growing into the vessel wall and reduces the risk of a restenosis, as well as the risk of a thrombosis. The controlled elution of rapamycin extends over a period of time from 1 to 12 months, preferably from 1 to 2 months after implantation.

### Active Agent Combinations

In the embodiments according to the invention rapamycin can be used also in combination with other active agents. As further antiproliferative, antimigrative, antiangiogenic, anti-inflammatoric, antiphlogistic, cytostatic, cytotoxic and/or antithrombotic active agents which promote the effect of rapamycin and/or its chemical as well as biological derivatives can be used: somatostatin, tacrolimus, roxithromycin, dunaimycin, ascomycin, bafilomycin, erythromycin, midecamycin, josamycin, concanamycin, clarithromycin, troleandomycin, folimycin, cerivastatin, simvastatin, lovastatin, fluvastatin, rosuvastatin, atorvastatin, pravastatin, pitavastatin, vinblastine, vincristine, vindesine, vinorelbine, etoposide, teniposide, nimustine, carmustine, lomustine, cyclophosphamide, 4-hydroxycyclophosphamide, estramustine, melphalan, ifosfamide, trofosfamide, chlorambucil, bendamustine, dacarbazine, busulfan, procarbazine, treosulfan, temozolomide, thiotepa, daunorubicin, doxorubicin, aclarubicin, epirubicin, mitoxantrone, idarubicin, bleomycin, mitomycin, dactinomycin, methotrexate, fludarabine, fludarabine-5′-dihydrogenephosphate, cladribine, mercaptopurine, thioguanine, cytarabine, fluorouracil, gemcitabine, capecitabine, docetaxel, carboplatin, cisplatin, oxaliplatin, amsacrine, irinotecan, topotecan, hydroxycarbamide, miltefosine, pentostatin, aldesleukin, tretinoin, asparaginase, pegaspargase, anastrozole, exemestane, letrozole, formestane, aminoglutethimide, adriamycin, azithromycin, spiramycin, cepharantin, 8-α-ergoline, dimethylergoline, agroclavin, 1-allylisurid, 1-allyltergurid, bromergurid, bromocriptin (ergotaman-3′,6′,18-trione, 2-bromo-12′-hydroxy-2′-(1-methylethyl)-5′-(2-methylpropyl)-, (5′ alpha)-), elymoclavin, ergocristin (ergotaman-3′,6′,18-trione, 12′-hydroxy-2′-(1-methylethyl)-5′-(phenylmethyl)-, (5′-alpha)-), ergocristinin, ergocornin (ergotaman-3′,6′,18-trione, 12′-hydroxy-2′,5′-bis(1-methylethyl)-, (5′-alpha)-), ergocorninin, ergocryptin (ergotaman-3′,6′,18-trione, 12′-hydroxy-2′-(1-methylethyl)-5′-(2-methylpropyl)-, (5′ alpha)-(9Cl)), ergocryptinin, ergometrin, ergonovin (ergobasin, INN: ergometrin, (8beta(S))-9,10-didehydro-N-(2-hydroxy-1-methylethyl)-6-methyl-ergoline-8-carboxamid), ergosin, ergosinin, ergotmetrinin, ergotamin (ergotaman-3′,6′,18-trione, 12′-hydroxy-2′-methyl-5′-(phenylmethyl)-, (5′-alpha)-(9Cl)), ergotaminin, ergovalin (ergotaman-3′,6′,18-trione, 12′-hydroxy-2′-methyl-5′-(1-methylethyl)-, (5′ alpha)-), lergotril, lisurid (CAS-No.: 18016-80-3,3-(9,10-didehydro-6-methylergolin-8alpha-yl)-1,1-diethyl carbamide), lysergol, lysergic acid (O-lysergic acid), lysergic acid amide (LSA, O-lysergic acid amide), lysergic acid diethylamide (LSD, O-lysergic acid diethylamide, INN: lysergamide, (8beta)-9,10-didehydro-N,N-diethyl-6-methyl-ergoline-8-carboxamide), isolysergic acid (D-isolysergic acid), isolysergic acid amide (D-isolysergic acid amide), isolysergic acid diethylamide (D-isolysergic acid diethylamide), mesulergin, metergolin, methergin (INN: methylergometrin, (8beta(S))-9,10-didehydro-N-(1-(hydroxymethyl)propyl)-6-methyl-ergoline-8-carboxamide), methylergometrin, methysergid (INN: methysergid, (8beta)-9,10-didehydro-N-(1-(hydroxymethyl)propyl)-1,6-dimethyl-ergoline-8-carboxamide), pergolid ((8beta)-8-((methylthio)methyl)-6-propyl-ergolin), protergurid and tergurid, celecoxip, thalidomid, Fasudil®, ciclosporin, smc proliferation inhibitor-2w, epothilone A and B, mitoxantrone, azathioprine, mycophenolatmofetil, c-myc-antisense, b-myc-antisense, betulinic acid, camptothecin, PI-88 (sulfated oligosaccharide), melanocyte-stimulating hormone (α-MSH), aktivated protein C, IL1-β-inhibitor, thymosine α-1, fumaric acid and its esters, calcipotriol, tacalcitol, lapachol, β-lapachone, podophyllotoxin, betulin, podophyllic acid 2-ethylhydrazide, molgramostim (rhuGM-CSF), peginterferon α-2b, lanograstim (r-HuG-CSF), filgrastim, macrogol, dacarbazin, basiliximab, daclizumab, selectin (cytokine antagonist) CETP inhibitor, cadherines, cytokinin inhibitors, COX-2 inhibitor, NFkB, angiopeptin, ciprofloxacin, camptothecin, fluoroblastin, monoclonal antibodies, which inhibit the muscle cell proliferation, bFGF antagonists, probucol, prostaglandins, 1,11-dimethoxycanthin-6-on, 1-hydroxy-11-methoxycanthin-6-on, scopolectin, colchicine, NO donors such as pentaerythritol tetranitrate and syndnoeimines, S-nitrosoderivatives, tamoxifen, staurosporine, β-estradiol, α-estradiol, estriol, estrone, ethinylestradiol, fosfestrol, medroxyprogesterone, estradiol cypionates, estradiol benzoates, tranilast, kamebakaurin and other terpenoids which are applied in the therapy of cancer, verapamil, tyrosine kinase inhibitors (tyrphostines), cyclosporine A, paclitaxel and its derivatives such as 6-α-hydroxy-paclitaxel, baccatin, taxotere, synthetically produced macrocyclic oligomers of carbon suboxide (MCS) and its derivatives as well as those obtained from native sources, mofebutazone, acemetacin, diclofenac, lonazolac, dapsone, o-carbamoylphenoxyacetic acid, lidocaine, ketoprofen, mefenamic acid, piroxicam, meloxicam, chloroquine phosphate, penicillamine, tumstatin, avastin, D-24851, SC-58125, hydroxychloroquine, auranofin, sodium aurothiomalate, oxaceprol, celecoxib, B-sitosterin, ademetionine, myrtecaine, polidocanol, nonivamide, levomenthol, benzocaine, aescin, ellipticine, D-24851 (Calbiochem), colcemid, cytochalasin A-E, indanocine, nocodazole, S 100 protein, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor-1, plasminogen activator inhibitor-2, antisense oligonucleotides, VEGF inhibitors, IGF-1, active agents from the group of antibiotics such as cefadroxil, cefazolin, cefaclor, cefotaxim, tobramycin, gentamycin, penicillins such as dicloxacillin, oxacillin, sulfonamides, metronidazol, antithrombotics such as argatroban, aspirin, abciximab, synthetic antithrombin, bivalirudin, coumadin, enoxaparin, desulfated and N-reacetylated heparin, tissue plasminogen activator, GpIIb/IIIa platelet membrane receptor, factor Xₐ inhibitor antibodies, interleukin inhibitors, heparin, hirudin, r-hirudin, PPACK, protamine, sodium salt of 2-methylthiazolidin-2,4-dicarboxylic acid, prourokinase, streptokinase, warfarin, urokinase, vasodilators such as dipyramidole, trapidil, nitroprussides, PDGF antagonists such as triazolopyrimidine and seramin, ACE inhibitors such as captopril, cilazapril, lisinopril, enalapril, losartan, thioprotease inhibitors, prostacyclin, vapiprost, interferon α, β and γ, histamine antagonists, serotonine blockers, apoptosis inhibitors, apoptosis regulators such as p65, NF-kB or Bcl-xL antisense oligonucleotides, halofuginone, nifedipine, tocopherol, vitamin B1, B2, B6 and B12, folic acid, tranilast, molsidomine, tea polyphenols, epicatechin gallate, epigallocatechin gallate, Boswellic acids and their derivatives, leflunomide, anakinra, etanercept, sulfasalazine, etoposide, dicloxacillin, tetracycline, triamcinolone, mutamycin, procainamid, D24851, SC-58125, retinoic acid, quinidine, disopyramide, flecamide, propafenone, sotalol, amidorone, natural and synthetically prepared steroids such as bryophyllin A, inotodiol, maquirosid A, ghalakinosid, mansonin, streblosid, hydrocortisone, betamethasone, dexamethasone, non-steroidal substances (NSAIDS) such as fenoprofen, ibuprofen, indomethacin, naproxen, phenylbutazone and other antiviral agents such as acyclovir, ganciclovir and zidovudine, antimycotics such as clotrimazole, flucytosine, griseofulvin, ketoconazole, miconazole, nystatin, terbinafine, antiprotozoal agents such as chloroquine, mefloquine, quinine, furthermore natural terpenoids such as hippocaesculin, barringtogenol-C21-angelate, 14-dehydroagrostistachin, agroskerin, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, hyptatic acid A, zeorin, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, 1,11-dimethoxycanthin-6-one, 1-hydroxy-11-methoxycanthin-6-one, scopoletin, taxamairin A and B, regenilol, triptolide, furthermore cymarin, apocymarin, aristolochic acid, anopterin, hydroxyanopterin, anemonin, protoanemonin, berberine, cheliburin chloride, cictoxin, sinococuline, bombrestatin A and B, cudraisoflavone A, curcumin, dihydronitidine, nitidine chloride, 12-beta-hydroxypregnadiene-3,20-dione, bilobol, ginkgol, ginkgolic acid, helenalin, indicine, indicine-N-oxide, lasiocarpine, inotodiol, glycoside 1a, podophyllotoxin, justicidin A and B, larreatin, malloterin, mallotochromanol, isobutyrylmallotochromanol, maquiroside A, marchantin A, maytansine, lycoridicin, margetine, pancratistatin, liriodenine, oxoushinsunine, aristolactam-All, bisparthenolidine, periplocoside A, ghalakinoside, ursolic acid, deoxypsorospermin, psychorubin, ricin A, sanguinarine, manwu wheat acid, methylsorbifolin, sphatheliachromen, stizophyllin, mansonine, strebloside, akagerine, dihydrousambarensine, hydroxyusambarine, strychnopentamine, strychnophylline, usambarine, usambarensine, berberine, liriodenine, oxoushinsunine, daphnoretin, lariciresinol, methoxylariciresinol, syringaresinol, umbelliferon, afromoson, acetylvismione B, desacetylvismione A, vismione A and B, and sulfur-containing amino acids such as cysteine as well as salts, hydrates, solvates, enantiomers, racemates, enantiomeric mixtures, diastereomeric mixtures, metabolites and mixtures of the above mentioned active agents.

The active agents are used separately or combined in the same or a different concentration. Especially preferred are active agents which have, besides their antiproliferative effect, further properties. Moreover, a combination with the active agents tacrolimus, paclitaxel and its derivatives, Fasudil®, vitronektin receptor antagonists, thalidomid, cyclosporin A, tergurid, lisurid, celecoxip, R-lys compounds and their derivatives/analogues as well as effective metabolites is preferred. Especially preferred is a combination of rapamycin with tergurid or rapamycin with lisurid or rapamycin with paclitaxel or rapamycin with an immunosuppressive such as cyclosporin A.

Especially preferred is an active agent combination of rapamycin with paclitaxel, derivatives of paclitaxel, especially the hydrophilic derivatives of paclitaxel, epothilon, tergurid or lisurid.

The active agent is preferably contained in a pharmaceutically active concentration from 0.001-10 mg per cm² of stent surface. Other active agents can be contained in a similar concentration in the same or in other layers, wherein it is preferred if the one or the further active agents are contained in a different layer than rapamycin.

### Polymers

If the active agent or active agent combination is not applied directly on the or into the stent, besides the hemocompatible conditioning of the surface with suitable hemocompatible substances of synthetic, semisynthetic and/or native origin, biostable and/or biodegradable polymers or polysaccharides can be used as carriers or as matrix.

As generally biologically stable and only slowly biologically degradable polymers can be mentioned: polyacrylic acid and polyacrylates such as polymethylmethacrylate, polybutylmethacrylate, polyacrylamide, polyacrylonitriles, polyamides, polyetheramides, polyethylenamine, polyimides, polycarbonates, polycarbourethanes, polyvinylketones, polyvinylhalogenides, polyvinylidenhalogenides, polyvinylethers, polyvinylaromates, polyvinylesters, polyvinylpyrollidones, polyoxymethylenes, polyethylene, polypropylene, polytetrafluoroethylene, polyurethanes, polyolefine elastomeres, polyisobutylenes, EPDM gums, fluorosilicones, carboxymethylchitosane, polyethylenterephthalate, polyvalerates, carboxymethylcellulose, cellulose, rayon, rayontriacetates, cellulosenitrates, celluloseacetates, hydroxyethylcellulose, cellulosebutyrates, celluloseacetatebutyrates, ethylvinylacetate copolymers, polysulfones, polyethersulfones, epoxy resins, ABS resins, EPDM gums, silicon prepolymers, silicones such as polysiloxanes, polyvinylhalogenes and copolymers, celluloseethers, cellulosetriacetates, chitosane and chitosane derivatives, polymerizable oils such as linseed oil and copolymers and/or mixtures of these substances.

As generally biologically degradable or resorbable polymers can be used e.g.: polyvalerolactones, poly-ε-decalactones, polylactides, polyglycolides, copolymers of the polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-one), poly-para-dioxanones, polyanhydrides such as polymaleic anhydrides, polyhydroxymethacrylates, fibrin, polycyanoacrylates, polycaprolactonedimethylacrylates, poly-β-maleic acid, polycaprolactonebutyl-acrylates, multiblock polymers such as from oligocaprolactonedioles and oligodioxanonedioles, polyetherester multiblock polymers such as PEG and poly(butyleneterephtalates), polypivotolactones, polyglycolic acid trimethyl-carbonates, polycaprolactone-glycolides, poly(g-ethylglutamate), poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcoholes, polyesteramides, glycolated polyesters, polyphosphoesters, polyphosphazenes, poly[p-carboxyphenoxy)propane], polyhydroxypentanoic acid, polyethyleneoxide-propyleneoxide, soft polyurethanes, polyurethanes having amino acid residues in the backbone, polyether esters such as polyethyleneoxide, polyalkeneoxalates, polyorthoesters as well as their copolymers, carrageenanes, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethylsulfate, albumin, hyaluronic acid, heparansulfates, heparin, chondroitinesulfate, dextran, B-cyclodextrines, copolymers with PEG and polypropyleneglycol, gummi arabicum, guar, gelatine, collagen, collagen-N-hydroxysuccinimide, lipids and lipoids, polymerizable oils having a low degree of cross-linking, modifications and copolymers and/or mixtures of the afore mentioned substances.

Preferred polymers as carriers for rapamycin or polymers for the incorporation of rapamycin are polylactides, polyglycolides, copolymers of polylactides and polyglycolides, polyhydroxybutyrates, polyhydroxymethacrylates, polyorthoesters, glycolated polyesters, polyvinylalcohols, polyvinylpyrrolidone, acrylamide-acrylic acid-copolymers, hyaluronic acid, heparanesulfate, heparin, chondroitinsulfate, dextrane, β-cyclodextrines, hydrophilically cross-linked dextrins, alginates, phospholipids, carbomers, cross-linked peptides and proteins, silicones, polyethyleneglycol (PEG), polypropyleneglycol (PPG), copolymers of PEG and PPG, collagen, polymerizable oils and waxes, as well as their mixtures and copolymers.

Moreover, polyesters, polylactids as well as copolymers of diols and esters or diols and lactids are preferred. For example, ethane-1,2-diol, propane-1,3-diol or butane-1,4-diol are used as diols.

According to the invention especially polyesters are used for the polymer layer. From the group of polyesters such polymers are preferred which have the following repeating units:

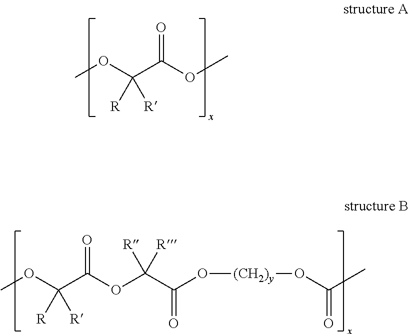

In the shown repeating units R, R′, R″ and R′″ represents an alkyl residue having 1 to 5 carbon atoms, especially methyl, ethyl, propyl, isopropyl, n-butyl, s-butyl, t-butyl, iso-butyl, n-pentyl or cyclopentyl and preferably methyl or ethyl. Y represents an integer from 1 to 9 and X represents the degree of polymerization. Especially preferred are the following polymers having the shown repeating units:

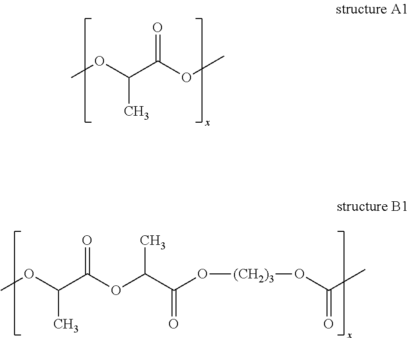

As further representatives of the resorbable polymers Resomer® shall be mentioned the poly(L-lactid)es having the general formula —(C₆H₈O₄)ₙ— such as L 210, L 210 S, L 207 S, L 209 S, the poly(L-lactid-co-D,L-lactid)es having the general formula —(C₆H₈O₄)ₙ— such as LR 706, LR 708, L 214 S, LR 704, the poly(L-lactid-co-trimethylcarbonat)es having the general formula —[(C₆H₈O₄)ₓ—(C₄H₆O₃)_{y}]ₙ— such as LT 706, the poly(L-lactid-co-glycolid)es having the general formula —[(C₆H₈O₄)ₓ—(C₄H₄O₄)_{y}]ₙ— such as LG 824, LG 857, the poly(L-lactid-co-ε-caprolacton)es having the general formula —[(C₆H₈O₄)ₓ—(C₆H₁₀O₂)_{y}]ₙ— such as LC 703, the poly(D,L-lactid-co-glycolid)es having the general formula —[(C₆H₈O₄)ₓ—(C₄H₄O₄)_{y}]ₙ— such as RG 509 S, RG 502 H, RG 503 H, RG 504 H, RG 502, RG 503, RG 504, the poly(D,L-lactid)es having the general formula —(C₆H₈O₄)ₙ— such as R 202 S, R 202 H, R 203 S and R 203 H. Resomer® 203 S represents the follower of the especially preferred polymer Resomer® R 203. The name Resomer® represents a high-tech product from the company Boehringer Ingelheim.

In principle, the use of resorbable polymers in the present invention is especially preferred. Moreover, homopolymers of lactic acid (polylactides) as well as polymers which are prepared from lactic and glycolic acid are preferred.

Surprisingly it was found that in the use of the resomers, polylactides, polymers of the structure A or A1, polymers of the structure B or B1 as well as the copolymers of lactic acid and glycolic acid (PLGAs) an elution of rapamycin is achieved which is advantageous for the healing. As it can be seen from the elution graph, a continuous constantly increasing elution of the active agent occurs within the first weeks, then the elution graph is steeper and the elution of rapamycin occurs more rapidly. This fact is of great advantage. In the first phase after a vessel dilatation a continuously increasing small amount of rapamycin is eluted which results in a moderate suppression of an overshooting inflammatory reaction, but does not suppress this necessary reaction. Then, after the first decisive weeks any increased proliferation reaction and still existing inflammatory parameters are curtailed by the more rapid elution of further amounts of rapamycin.

### Rapamycin and PVA

Thus, an advantageous embodiment of the present invention is a rapamycin-coated stent which has a pure active agent layer of rapamycin on the stent surface that is covered by a protective layer of a bioresorbable polymer and preferably by a protective layer of a resomer, polyvinylalcohol (PVA), polylactides, polymers of the structure A1, polymers of the structure A2 as well as the copolymers of lactic acid and glycolic acid (PLGA) or mixtures of the above mentioned polymers. Further examples for bioresorbable polymers are mentioned below. The properties of the topcoat determine the elution of the subjacent rapamycin and are also substantially responsible for the stability and therewith the shelf life of the coated stent. Thus, the beginning of the elution can be altered temporarily while the elution itself is strongly accelerated such that in a shorter time more rapamycin is eluted. For example, in using polyvinyl alcohol as protective layer rapamycin is completely eluted after three days. By adding rapamycin into the topcoat an even higher dosing can be achieved.

The pure rapamycin layer is preferably completely covered by a bioresorbable, i.e. biologically degradable polymer layer.

In another preferred embodiment a hemocompatible coating can be directly on the stent surface and under the pure active agent layer of rapamycin. As hemocompatible substances the ones mentioned herein can be used, wherein the below mentioned heparin derivatives or chitosan derivatives of the general formulas Ia or Ib as well as the below described oligo- and polysaccharides which contain over 95% the sugar units N-acylglucosamine and uronic acid (preferred glucuronic acid and iduronic acid) or N-acylgalactosamine and uronic acid are preferred. Thus, a preferred embodiment is a stent with a preferably covalently bound hemocompatible coating and a pure rapamycin layer thereon with an external biodegradable protective layer.

In another preferred embodiment the stent is provided with a pure rapamycin layer whereon a bioresorbable layer is applied, wherein a further active agent layer of rapamycin is applied to this bioresorbable layer which in turn is provided with a biologically degradable layer. Thus, stents are preferred which have an alternating series of layers of rapamycin and bioresorbable layer, wherein between 3 to 10 layers are possible. Normally, a protective layer is preferred as external layer, wherein the external layer can be also a rapamycin layer. For the bioresorbable layers the same bioresorbable polymers can be used or for the generation of a differently rapid degradation of the single layers also different bioresorbable polymers can be used, wherein it is preferred when the degradation rate increases from the external to the most inner layer or from the most inner layer to the external layer. Also in the multi-layer systems a lower hemocompatible layer can be used which is preferably covalently bound to the stent surface.

Moreover, also coated catheter balloons are preferred which have a pure active agent layer of rapamycin and an adjacent protective layer of a bioresorbable polymer. For catheter balloons two-layer systems are preferred.

In another embodiment a contrast agent or contrast agent analogue (contrast agent-like matter) is used instead of the bioresorbable polymer. As contrast agents the below mentioned compounds can be used.

Thus, catheter balloons or stents are preferred which have a pure rapamycin layer and an adjacent contrast agent layer.

Moreover, the stents can have also an alternating sequence of rapamycin layers an contrast agent layers and optionally the stent can have a hemocompatible layer which is preferably covalently bound to the stent surface of the herein mentioned hemocompatible substances.

The rapamycin layer and the contrast agent layer or the layer of bioresorbable polymer are preferably applied to the stent or the catheter balloon in the spraying method, wherein the catheter balloon can be coated in the expanded as well as the compressed state.

Suchlike two-layer systems or multi-layer systems on a stent or suchlike two-layer systems on a catheter balloon are manufactured by spraying the preferably uncoated or hemocompatible layer-coated surface of the stent or the preferably uncoated surface of the catheter balloon with a rapamycin-containing solution and spraying the as-prepared active agent layer preferably after drying with a solution of the polymer of the protective layer in a polar solvent which has a water content of less than 50% by volume, preferably less than 40% by volume and especially preferred less than 30% by volume.

Suitable solvents for the polymer especially for the hydrophilic polymer of the protective layer are hydrophilic solvents and preferably acetone, butanone, pentanone, tetrahydrofuran (THF), acetic acid ethylester (ethylacetate), methanol, ethanol, propanol, iso-propanol as well as mixtures of the above mentioned solvents which have a water content of 1% to 50% by volume, preferably 5% to 40% by volume and especially preferred of 10% to 30% by volume.

As-manufactured coating systems are superior to the known coating systems with respect to stability of rapamycin and elution kinetics.

### Rapamycin and Polysulfone

The use of polysulfones has the decisive advantage that the polysulfone itself has very good hemocompatible properties and is moreover biostable, i.e. a permanent coating of the stent surface is present, which is hemocompatible and is not degraded biologically and also functions as active agent carrier for rapamycin.

Polysulfone has the decisive advantage that it does not create a risk of late thromboses which other coating systems could have whereby polymer-coated drug-eluting stents have made negative headlines in the past.

Polysulfone as biologically stable coating which is not or only extremely slowly degraded after implantation of the stent in the body of the patient has on the contrary the disadvantage that it does not elute rapamycin to a sufficient extent. To guarantee a sufficient elution of rapamycin the polysulfone is added according to the invention a certain content of a hydrophilic or methanol-swellable polymer.

By admixing of hydrophilic polymers different methods can be achieved for the targeted application of rapamycin or combinations with other preferred active agents. While in a concentration of 0.1% to 1% the hydrophilic polymer is dispersed in the polysulfone matrix in form of small pores, the permeability of the polysulfone increases with increasing content of the hydrophilic polymer such that after a critical concentration also channels are formed which get up to the surface. The critical concentration for the formation of channels depends on the hydrophilic polymer from 3% to 8% by weight with respect to the weight of the total coating or the weight of polysulfone and hydrophilic polymer.

If an as-coated stent is in a vessel it comes into contact with the aqueous medium such as body fluids and the hydrophilic active agent absorbs liquid. Thereby, an overpressure is formed within the channels and the active agent reservoirs such that the elution of the also hydrophilic active agent occurs in the form of an “injection” targetedly to and into the vessel wall. Additionally, the non-swelling matrix can also contain rapamycin or another preferred active agent or a combination of rapamycin and another active agent and therewith promote the long-term regulation of the healing process.

Examples of hydrophilic polymers are given below and are also well known to a skilled person. Herein, such polymers are referred to as hydrophilic polymers which are soluble or at least swellable in methanol. Swellable means the ability of the polymer to absorb methanol into the polymer framework whereby the volume of the polymer material increases.

To create a suitable elution kinetics of rapamycin from the polysulfone the polysulfone is added 0.1% to 50% by weight, preferably 1.0% to 30% by weight and especially preferred 5% to 20% by weight of a methanol-swellable polymer. Basically, the tendency for channel formation in the polysulfone coating increases with increasing content of hydrophilic or methanol-swellable polymer.

Suitable methanol-swellable polymers are listed below. Suitable examples are the following mixtures:
polysulfone having 2% by weight of polyvinylpyrollidone (PVP)
polysulfone having 11% by weight of glycerine
polysulfone having 8% by weight of polyethyleneglycol
polysulfone having 6% by weight of polyvinylalcohol
polysulfone having 5% by weight of polyhydroxyethyl-methacrylate
polysulfone having 7% by weight of polyacrylamide
polysulfone having 4% by weight of polylactide
polysulfone having 9% by weight of polyesteramide
polysulfone having 1% by weight of chondroitinsulfate
polysulfone having 8% by weight of polyhydroxybutyrate

The methanol-swellable polymer forms after implantation of the stent cracks and channels in the polysulfone coating which serve for eluting rapamycin and thus result despite of a biostable polysulfone coating in a proper elution rate of rapamycin after stent implantation. Suitable polysulfones for the biostable coating are discussed in detail more below.

The stents according to the invention are manufactured by providing a preferably uncoated stent which is sprayed with a solution of polysulfone and rapamycin and the methanol-swellable or hydrophilic polymer in a suitable solvent (methylenechloride (dichloromethane), methylacetate, trichloroethylene:methylenechloride 1:1 (v/v), chloroform, dimethylformamide, ethanol, methanol, acetone, THF, ethylacetate, etc.). The spraying process can be continuous or sequential with drying steps between the spraying steps or the coating can also be applied in the dipping method, brushing method or plasma method.

In this embodiment preferably combinations of polysulfone with the hydrophilic polymers which are soluble in the same organic solvents as polysulfone are used. Thus, a skilled person can easily determine a suitable co-polymer for the polysulfone by determining the solution behavior of the selected polysulfone (suitable and also preferred polysulfones are described in detail more below) and then checking if the selected co-polymer has similar solution properties. The solution properties are to be considered similar when the dissolved amount of polysulfone K per volume unit solvent (e.g. per 1 ml) to the dissolved amount J of co-polymer per same volume unit of solvent (e.g. 1 ml) meets 0.5K<J<2K.

Examples of suitable hydrophilic or methanol-swellable polymers are selected from the group comprising or consisting of: polyvinylpyrrolidone, polylactide, pectines, glycerin, polyethylene glycol, polypropylene glycol, polyvinyl alcohol, polyhydroxyethyl methacrylates, polyacrylamide, polyvalerolactones, poly-ε-decalactones, polylactonic acid, polyglycolic acid, polylactides, polyglycolides, copolymers of polylactides and polyglycolides, poly-ε-caprolactone, polyhydroxybutanoic acid, polyhydroxybutyrates, polyhydroxyvalerates, polyhydroxybutyrate-co-valerates, poly(1,4-dioxane-2,3-diones), poly(1,3-dioxane-2-ones), poly-para-dioxanones, polyanhydrides such as polymaleic anhydrides, fibrin, polycyanoacrylates, polycaprolactonedimethylacrylates, poly-β-maleic acid, polycaprolactone butylacrylates, multiblock polymers such as from oligocaprolactonedioles and oligodioxanonedioles, polyether ester multiblock polymers such as PEG and polybutylene terephthalate, polypivotolactones, polyglycolic acid trimethyl-carbonates, polycaprolactone-glycolides, poly-g-ethylglutamate, poly(DTH-iminocarbonate), poly(DTE-co-DT-carbonate), poly(bisphenol-A-iminocarbonate), polyorthoesters, polyglycolic acid trimethyl-carbonates, polytrimethylcarbonates, polyiminocarbonates, poly(N-vinyl)-pyrrolidone, polyvinylalcohols, polyesteramides, glycolated polyesters, polyphosphoesters, polyphosphazenes, poly[p-carboxyphenoxy)propane], polyhydroxypentanoic acid, polyanhydrides, polyethyleneoxide-propyleneoxide, soft polyurethanes, polyurethanes with amino acid residues in the backbone, polyether esters, polyethyleneoxide, polyalkeneoxalates, polyorthoesters as well as copolymers thereof, lipids, carrageenans, fibrinogen, starch, collagen, protein based polymers, polyamino acids, synthetic polyamino acids, zein, modified zein, polyhydroxyalkanoates, pectic acid, actinic acid, modified and non modified fibrin and casein, carboxymethyl sulfate, albumin, hyaluronic acid, chitosan and its derivatives, chondroitine sulfate, dextran, B-cyclodextrins, copolymers with PEG and polypropylene glycol, gum arabic, guar, gelatin, collagen, collagen-N-hydroxysuccinimide, lipids, phospholipids, modifications and copolymers and/or mixtures of the above mentioned substances.

Especially preferred are polyvinylpyrrolidone, polyethyleneglycol, polylactides and -glycolides and their copolymers. Preferably used as solvent are chloroform, dichloromethane and methylenechloride, acetone and methylacetate, wherein especially chloroform is preferred. The content of rapamycin in the coating solution (preferred spraying solution) is between 60% and 10% by weight, preferably between 50% and 20% by weight, especially preferred between 40% and 30% by weight with respect to the weight of the total coating.

Further it is preferred to use anhydrous, i.e. dried, solvents or solvents having a water content of less than 2% by volume, preferably less than 1% by volume and especially preferred less than 0.2% by volume. Additionally, it was found as advantageous to perform the coating under exclusion of light to prevent a decomposition of rapamycin and to have a better control of the amount of active rapamycin in the coating. Further, it is advantageous to perform the coating in a dry, i.e. anhydrous, environment and to use as carrier gas for the coating an inert gas such as nitrogen or argon instead of air. Thus, the present invention also relates to coated stents which are coated according to the conditions mentioned above.

### Rapamycin and PLGA

Another preferred embodiment is a polymeric PLGA carrier for rapamycin on stents. PLGA refers to a block copolymer of polylactide and polyglycolic acid (polyglycolide) having the following general formula:

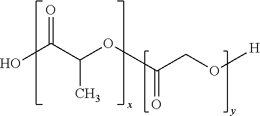

wherein
x represents the number of lactic acid units and y represents the number of glycolic acid units.

For manufacturing this coating rapamycin and PLGA is dissolved in a suitable solvent (chloroform, methanol, acetone, THF, ethylacetate, etc.) and sprayed on the preferably uncoated stent surface.

Instead of using a preferably uncoated stent surface the stent surface can be also provided with a preferably covalently bound hemocompatible layer on which the rapamycin-PLGA mixture is applied to.

By means of this embodiment the administration of rapamycin to the target site can be achieved in a special and surprisingly easy way, where it can be effective in a targeted and dosage-controlled way. As already described at the beginning, it is important that the active agent used does not repress the inflammatory reactions which are important for the process of wound healing to strongly because therewith the necessary condition for the starting healing process is suppressed. Rather, it is important to possibly reduce the inflammatory processes to the implantation. This basic demand is excellently solved by this coating form. Rapamycin as inflammatory inhibitor and immunosuppressive interacts with these processes but does not suppress them.

After a suchlike moderate regulation of the inflammatory processes the eluted rapamycin dosage is continuously increased until the complete degradation of the polymer. This is clarified by the elution graph of FIG. 4. Two inclinations can be seen in the graph, wherein the first phase has a smaller elution than the second phase. With the second increased elution of rapamycin the next important aspect of restenosis prophylaxis is considered. On the one hand, possibly still existing inflammatory regions in the tissue a repelled, on the other hand, now the antiproliferative effect of rapamycin gets important by regulation of the proliferation of smooth muscle cells in the wound region. Ideally, the stent surface on the luminal site should be covered by a layer of endothelial cells. But the increased proliferation activity of smooth muscle cells does not permit such a layer and covers the stent by forming fibrotic tissue. Finally, this results in a renewed disease. The accelerated elution of rapamycin regulates the proliferation activity of smooth muscle cells and reduces it to a normal and necessary extent of wound sealing.

If additionally the surface of the stent, as already mentioned, is provided with a covalently bound hemocompatible layer then it is additionally guaranteed that during the slow degradation of PLGA in the following weeks after implantation the coagulation system does not detect exposed regions as a foreign surface. Thus, an athrombogenic surface is provided which provides for a complete masking of the stent surface.

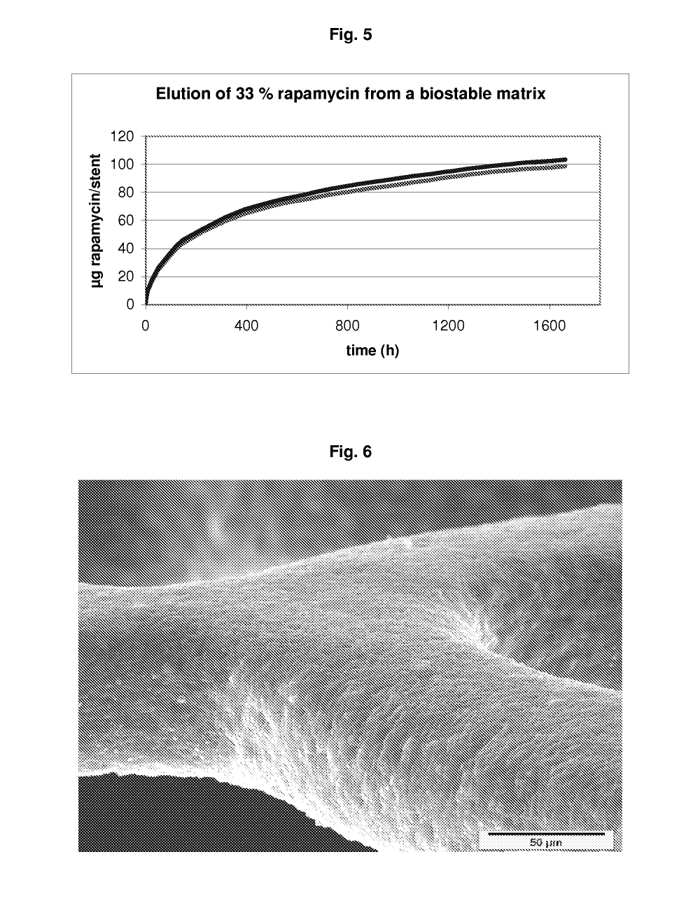

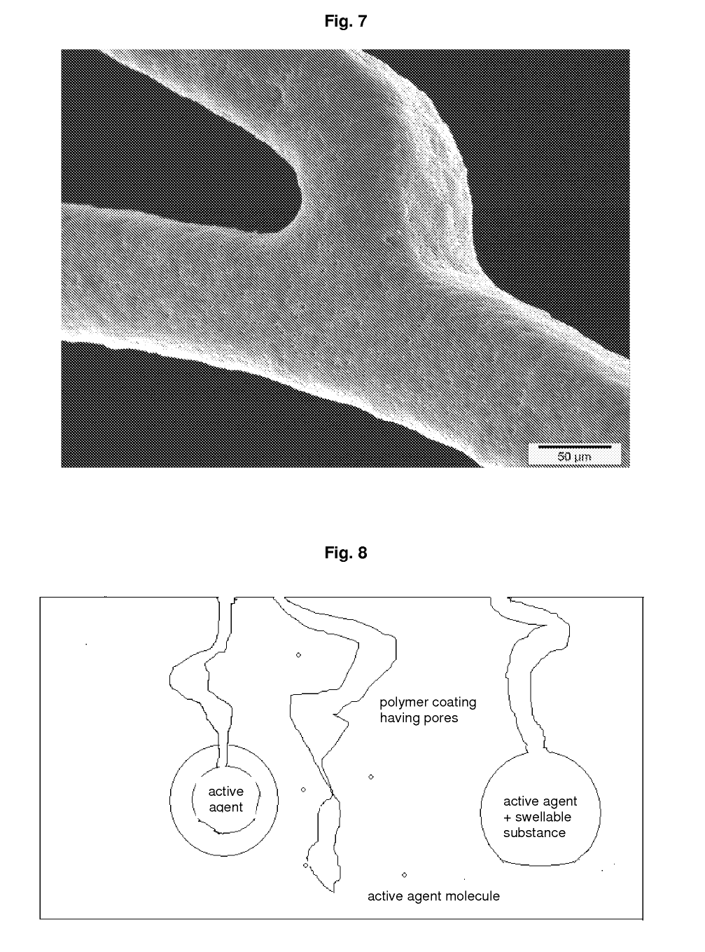

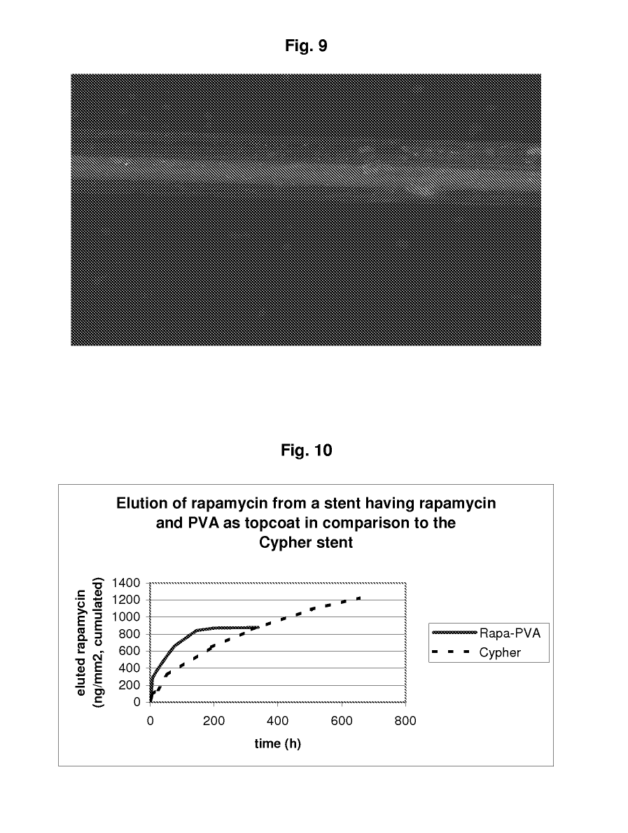

This unusual and especially advantageous elution kinetics shown in FIG. 4 could be obtained until now only with a system of PLGA as polymer carrier for rapamycin while the normal elution kinetics is shown in FIG. 5 and occurs in the other carrier systems, especially in the biostable carrier systems.

The PLGA-rapamycin coating according to the invention is obtained by dissolving PLGA and preferably PLGA (50/50) together with rapamycin in a suitable polar solvent (such as methylenechloride (dichloromethane), methylacetate, trichloroethylene:methylenechloride 1:1 (v/v), chloroform, dimethylformamide, ethanol, methanol, acetone, THF, ethylacetate, etc.) and spraying the preferably uncoated or hemocompatibly coated stent surface with this solution. The spraying process can be continuous or sequential with drying steps between the spraying steps or the coating can also be applied in the dipping method, brushing method or plasma method.

The content of rapamycin in the coating solution (preferred spraying solution) is between 60% and 10% by weight, preferably between 50% and 20% by weight, especially preferred between 40% and 30% by weight with respect to the weight of the total coating.

Further it is preferred to use anhydrous, i.e. dried, solvents or solvents having a water content of less than 2% by volume, preferably less than 1% by volume and especially preferred less than 0.2% by volume Additionally, it was found as advantageous to perform the coating under exclusion of light to prevent a decomposition of rapamycin and to have a better control of the amount of active rapamycin in the coating. Further, it is advantageous to perform the coating in a dry, i.e. anhydrous, environment and to use as carrier gas for the coating an inert gas such as nitrogen or argon instead of air. Thus, the present invention also relates to coated stents which are coated according to the conditions mentioned above.

### Balloon Coating

Another preferred embodiment is the coating of balloon catheters with rapamycin.

In PTCA the narrowed site is dilated, if necessary more than two times, for a short period of 1-3 minutes by means of the expandable balloon at the end of the catheter. The vessel walls have to be over-dilated such that the narrowing is eliminated. From this procedure micro-fissures result in the vessel walls which extend up to the adventitia. After removal of the catheter the injured vessel is left alone such that the healing process is demanded a more or less high-grade performance in dependence of the inflicted grade of injury which results from the dilatation duration, the dilatation repeats and the dilatation grade. This can be seen in the high reocclusion rate after PTCA. However, the utilization of PTCA has advantages in comparison to the stent, not only because in this way after the procedure of the treatment a foreign body is never present in the organism as additional stress or initiator for after-effects such as restenosis.

Also here rapamycin is well suitable due to its versatile mechanism of action. However, it has to be guaranteed that during PTCA the hydrophilic active agent is not lost or prematurely blistered in the dilatation.

Therefore, a method exists in which rapamycin or a combination with other active agents can be applied to a balloon and a targeted active agent amount can be absorbed by the vessel wall during the contacting time of up to several minutes.

Therefore, rapamycin is dissolved in a suitable organic solvent and applied to the balloon by means of spraying or pipetting method. Additionally, adjuvants are added to the rapamycin solution which either guarantee the visualization of the catheter or function as so-called transport mediators and promote the absorption of the active agent into the cell. These are comprised of vasodilators which comprise endogeneous substances such as kinins, e.g. bradykinin, kallidin, histamine or NOS-synthase which releases from L-arginin the vasodilatatory NO. Substances of herbal origin such as the extract of gingko biloba, DMSO, xanthones, flavonoids, terpenoids, herbal and animal dyes, food colorants, NO-releasing substances such as pentaerythrytiltetranitrate (PETN), contrast agents and contrast agent analogues belong also to these adjuvants or as such can be synergistically used as active agent.

Further substances to be mentioned are 2-pyrrolidon, tributyl- and triethylcitrate and their acetylated derivatives, bibutylphthalate, benzoic acid benzylester, diethanolamine, diethylphthalate, isopropylmyristate and -palmitate, triacetin etc.

Especially preferred are DMSO, iodine-containing contrast agents, PETN, tributyl- and triethylcitrate and their acetylated derivatives, isopropylmyristate and -palmitate, triacetin and benzoic acid benzylester.

Depending of the target site of a catheter a polymer matrix is necessary. Therewith, the premature blistering of a pure active agent layer is prevented. Biostable and biodegradable polymers can be used which are listed below. Especially preferred are polysulfones, polyurethanes, polylactides and glycolides and their copolymers.

### Hemocompatible Coating

Additionally, the stent surface can be provided with an athrombogenic or inert or biocompatible surface which guarantees that in the decrease of the active agent's influence and the degradation of the matrix no reactions occur on the existing foreign surface which in the long-term could also result in a reocclusion of the blood vessel. The hemocompatible layer which directly covers the stent is preferably comprised of heparin of native origin as well as synthetically prepared derivatives of different sulfation degrees and acylation degrees in the molecular weight range of the pentasaccharide which is responsible for the antithrombotic effect, up to the standard molecular weight of the commercially available heparin, heparansulfates and its derivatives, oligo- and polysaccharides of the erythrocytic glycocalix which perfectly represent the antithrombogenic surface of the erythrocytes because here contrary to phosphorylcholine the actual contact of blood and erythrocyte surface occurs, oligosaccharides, polysaccharides, completely desulfated and N-reacetylated heparine, desulfated and N-reacetylated heparine, N-carboxymethylated and/or partially N-acetylated chitosan, polyacrylic acid, polyvinylpyrrolidone and polyethyleneglycol and/or mixtures of these substances. These stents having a hemocompatible coating are manufactured by providing common normally uncoated stents and applying preferably covalently a hemocompatible layer which permanently masks the surface of the implant after drug elution and therewith after the decrease of the active agent's influence and the degradation of the matrix. Thus, this hemocompatible coating is also directly applied to the stent surface.

Thus, a preferred embodiment of the present invention relates to a stent of any material the surface of which is masked by the application of the glycocalix constituents of blood cells, esothelial cells or mesothelial cells. The glycocalix is the external layer of e.g. blood cells, esothelial cells or mesothelial cells due to which these cells are blood-acceptable (hemocompatible). The constituents of this external layer (glycocalix) of blood cells, esothelial cells and/or mesothelial cells is preferably enzymatically separated from the cell surface, separated from the cells and used as coating material for the stents. This glycocalix constituents are i.a. comprised of oligosaccharide, polysaccharide and lipid moieties of the glycoproteins, glycolipids and proteoglycanes as well as glycophorines, glycosphingolipids, hyaluronic acids, chondroitinsulfates, dermatansulfates, heparansulfates as well as keratansulfates. Methods for the isolation and use of these substances as coating materials are described in detail in the European Patent EP 1 152 778 B1 to the founders of the Hemoteq GmbH, Dr. Michael Hoffmann and Dipl.-Chem. Roland Horres. The covalent binding is achieved as in the case of hemoparin (see Example No. 9, 14 in the examples).

Further preferred embodiments have a most lower hemocompatible coating which is directly applied on the stent surface of desulfated and N-reacetylated heparin and/or N-carboxymethylated and/or partially N-acetylated chitosan. These compounds as well as the glycocalix constituents have already proved themselves in several studies as a very good hemocompatible coating and render the stent surface blood-acceptable after the adjacent active agent and/or carrier layers have been removed or biologically degraded. Suchlike especially preferred materials for the coating of the stent surface are disclosed in the European Patent No. EP 1 501 565 B1 of the Hemoteq AG. To this lower hemocompatible layer one or more active agent layers and/or active agent-free or active agent-containing carrier or polymer layers are applied.

These heparin derivatives or chitosan derivatives are polysaccharides of the general formula Ia

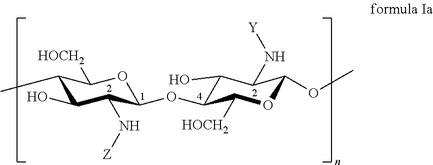

as well as structurally very similar polysaccharides of the general formula Ib

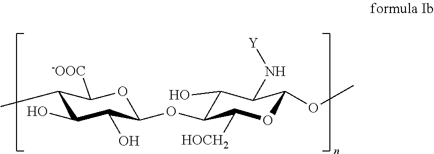

The polysaccharides according to formula Ia have molecular weights from 2 kD to 400 kD, preferably from 5 kD to 150 kD, more preferably from 10 kD to 100 kD, and especially preferred from 30 kD to 80 kD. The polysaccharides according to formula Ib have molecular weights from 2 kD to 15 kD, preferably from 4 kD to 13 kD, more preferably from 6 kD to 12 kD, and especially preferred from 8 kD to 11 kD. The variable n is an integer ranging from 4 to 1,050. Preferably, n is an integer from 9 to 400, more preferably from 14 to 260, and especially preferred an integer between 19 and 210.

The general formulas Ia and Ib represent a disaccharide, which is to be considered as a basic unit of the polysaccharide according to the invention and forms the polysaccharide by joining said basic unit to another one n times. Said basic unit comprising two sugar molecules does not intend to suggest that the general formulas Ia and Ib only relate to polysaccharides having an even number of sugar molecules. Of course, the general formula Ia and the formula Ib also comprise polysaccharides having an uneven number of sugar units. Hydroxy groups are present as terminal groups of the oligosaccharides or polysaccharides.

The groups Y and Z represent independently of each other the following chemical acyl or carboxyalkyl groups:

—CHO, —COCH₃, —COC₂H₅, —COC₃H₇, —COC₄H₉, —COC₅H₁₁, —COCH(CH₃)₂, —COCH₂CH(CH₃)₂, —COCH(CH₃)C₂H₅, —COC(CH₃)₃, —CH₂COO⁻, —C₂H₄COO⁻, —C₃H₆COO⁻, —C₄H₈COO⁻.

Preferred are the acyl groups —COCH₃, —COC₂H₅, —COC₃H₇ and the carboxyalkyl groups —CH₂COO⁻, —C₂H₄COO⁻, —C₃H₆COO⁻. More preferred are the acetyl and propanoyl groups and the carboxymethyl and carboxyethyl groups. Especially preferred are the acetyl group and the carboxymethyl group.

In addition, it is preferred that the group Y represents an acyl group, and the group Z represents a carboxyalkyl group. It is more preferred if Y is a group —COCH₃, —COC₂H₅, or —COC₃H₇, and especially —COCH₃. Moreover, it is further preferred if Z is a carboxyethyl or carboxymethyl group, wherein the carboxymethyl group is especially preferred.

The disaccharide basic unit shown by formula Ia comprises each a substituent Y and a further group Z. This is to make clear that the polysaccharide according to the invention comprises two different groups, namely Y and Z. It is important to point out here that the general formula Ia should not only comprise polysaccharides containing the groups Y and Z in a strictly alternating sequence, which would result from putting the disaccharide basic units one next to the other, but also polysaccharides carrying the groups Y and Z in a completely random sequence at the amino groups. Furthermore, the general formula Ia should also comprise such polysaccharides which contain the groups Y and Z in different numbers. Ratios of the number of Y groups to the number of X groups can be between 70%:30%, preferably between 60%:40%, and especially preferred between 45%:55%. Especially preferred are polysaccharides of the general formula Ia carrying on substantially half of the amino groups the Y residue and on the other half of the amino groups the Z residue in a merely random distribution. The term “substantially half” means exactly 50% in the most suitable case but should also comprise the range from 45% to 55% and especially 48% to 52% as well.

Preferred are the compounds of the general formula Ia, wherein the groups Y and Z represent the following:

| | | |
|---|---|---|
| Y = —CHO | and | Z = —C₂H₄COO⁻ |
| Y = —CHO | and | Z = —CH₂COO⁻ |
| Y = —COCH₃ | and | Z = —C₂H₄COO⁻ |
| Y = —COCH₃ | and | Z = —CH₂COO⁻ |
| Y = —COC₂H₅ | and | Z = —C₂H₄COO⁻ |
| Y = —COC₂H₅ | and | Z = —CH₂COO⁻ |

Especially preferred are the compounds of the general formula Ia, wherein the groups Y and Z represent the following:

| | | |
|---|---|---|
| Y = —CHO | and | Z = —C₂H₄COO⁻ |
| Y = —COCH₃ | and | Z = —CH₂COO⁻ |

Preferred are the compounds of the general formula Ib, wherein Y is one of the following groups: —CHO, —COCH₃, —COC₂H₅ or —COC₃H₇. Further preferred are the groups —CHO, —COCH₃, —COC₂H₅ and especially preferred is the group —COCH₃.

The compounds of the general formula Ib contain only a small amount of free amino groups. Because of the fact that with the ninhydrine reaction free amino groups could not be detected anymore, due to the sensitivity of this test it can be concluded that less than 2%, preferably less than 1% and especially preferred less than 0.5% of all —NH—Y groups are present as free amino groups, i.e. within this low percentage of the —NH—Y groups Y represents hydrogen.

Because polysaccharides of the general formulas Ia and Ib contain carboxylate groups and amino groups, the general formulas Ia and Ib cover also alkali as well as alkaline earth metal salts of the corresponding polysaccharides. Alkali metal salts like the sodium salt, the potassium salt, the lithium salt or alkaline earth metal salts like the magnesium salt or the calcium salt can be mentioned. Furthermore, with ammonia, primary, secondary, tertiary and quaternary amines, pyridine and pyridine derivatives ammonium salts, preferably alkylammonium salts and pyridinium salts can be formed. Among the bases, which form salts with the polysaccharides, are inorganic and organic bases as for example NaOH, KOH, LiOH, CaCO₃, Fe(OH)₃, NH₄OH, tetraalkylammonium hydroxide and similar compounds.

The compounds according to the invention of the general formula Ib can be prepared from heparin or heparansulfates by first substantially complete desulfation of the polysaccharide and subsequently substantially complete N-acylation. The term “substantially completely desulfated” refers to a desulfation degree of above 90%, preferred above 95% and especially preferred above 98%. The desulfation degree can be determined according to the so called ninhydrin test which detects free amino groups. The desulfation takes place to the extent that with DMMB (dimethylmethylene blue) no color reaction is obtained. This color test is suitable for the detection of sulfated polysaccharides but its detection limit is not known in technical literature. The desulfation can be carried out for example by pyrolysis of the pyridinium salt in a solvent mixture. Especially a mixture of DMSO, 1,4-dioxane and methanol has proven of value.

Heparansulfates as well as heparin were desulfated via total hydrolysis and subsequently reacylated. Thereafter the number of sulfate groups per disaccharide unit (S/D) was determined by ¹³C-NMR. The following table 1 shows these results on the example of heparin and desulfated, reacetylated heparin (Ac-heparin).

TABLE 1
Distribution of functional groups per disaccharide unit on the
example of heparin and Ac-heparin as determined by
¹³C-NMR-measurements.

| | 2-S | 6-S | 3-S | NS | N-Ac | NH₂ | S/D |
|---|---|---|---|---|---|---|---|
| Heparin | 0.63 | 0.88 | 0.05 | 0.90 | 0.08 | 0.02 | 2.47 |
| Ac-heparin | 0.03 | 0 | 0 | 0 | 1.00 | — | 0.03 |

2-S, 3-S, 6-S: sulfate groups in position 2, 3 or 6
NS: sulfate groups on the amino groups
N-Ac: acetyl groups on the amino groups
NH₂: free amino groups
S/D: sulfate groups per disaccharide unit

A sulfate content of about 0.03 sulfate groups/disaccharide unit (S/D) in the case of Ac-heparin in comparison with about 2.5 sulfate groups/disaccharide unit in the case of heparin was reproducibly obtained.

These compounds of the general formulas Ia and Ib have a content of sulfate groups per disaccharide unit of less than 0.2, preferred less than 0.07, more preferred less than 0.05 and especially preferred less than 0.03 sulfate groups per disaccharide unit.

Substantially completely N-acylated refers to a degree of N-acylation of above 94%, preferred above 97% and especially preferred above 98%. The acylation runs in such a way completely that with the ninhydrin reaction for detection of free amino groups no colour reaction is obtained anymore. As acylation agents are preferably used carboxylic acid chlorides, -bromides or -anhydrides. Acetic anhydride, propionic anhydride, butyric anhydride, acetic acid chloride, propionic acid chloride or butyric acid chloride are for example suitable for the synthesis of the compounds according to the invention. Especially suitable are carboxylic anhydrides as acylation agents.

In addition, the invention discloses oligosaccharides and/or polysaccharides for the hemocompatible coating of surfaces. Preferred are polysaccharides within the molecular weight limits mentioned above. One of the remarkable features of the oligosaccharides and/or polysaccharides used is that they contain large amounts of the sugar unit N-acylglucosamine or N-acylgalactosamine. This means that 40% to 60%, preferred 45% to 55% and especially preferred 48% to 52% of the sugar units are N-acylglucosamine or N-acylgalactosamine, and substantially the remaining sugar units each have a carboxyl group. Thus, usually more than 95%, preferably more than 98%, of the oligosaccharides and/or polysaccharides consist of only two sugar units, one sugar unit carrying a carboxyl group and the other one an N-acyl group.

One sugar unit of the oligosaccharides and/or polysaccharides is N-acylglucosamine or N-acylgalactosamine, preferably N-acetylglucosamine or N-acetylgalactosamine, and the other one is an uronic acid, preferably glucuronic acid and iduronic acid.

Preferred are oligosaccharides and/or polysaccharides substantially consisting of the sugar glucosamine or galactosamine, substantially half of the sugar units carrying an N-acyl group, preferably an N-acetyl group, and the other half of the glucosamine units carrying a carboxyl group directly bonded via the amino group or bonded via one or more methylenyl groups. These carboxylic acid groups bonded to the amino group are preferably carboxymethyl or carboxyethyl groups. Furthermore, oligosaccharides and/or polysaccharides are preferred, wherein substantially half of said oligosaccharides and/or polysaccharides, i.e. 48% to 52%, preferred 49% to 51% and especially preferred 49.5% to 50.5% consists of N-acylglucosamine or N-acylgalactosamine, preferably of N-acetylglucosamine or N-acetylgalactosamine, and substantially the other half thereof consists of an uronic acid, preferably glucuronic acid and iduronic acid. Especially preferred are oligosaccharides and/or polysaccharides showing a substantially alternating sequence (despite of the statistical error in the alternating junction) of the two sugar units. The rate of maljunctions should be under 1%, preferably 0.1%.

Surprisingly, it has been shown that, for the uses according to the invention, especially substantially desulfated and substantially N-acylated heparin as well as partially N-carboxyalkylated and N-acylated chitosan as well as desulfated and substantially N-acylated dermatansulfate, chondroitinsulfate and hyaluronic acid which is reduced in its chain length are especially suitable. Especially N-acetylated heparin and partially N-carboxymethylated and N-acetylated chitosan are suitable for the hemocompatible coating.

The desulfation degrees and acylation degrees defined by the term “substantially” have been defined already more above. The term “substantially” is intended to make clear that statistical deviations have to be taken into consideration. A substantially alternating sequence of the sugar units means that, as a rule, two equal sugar units are not bonded to each other, but does not completely exclude such a maljunction. Correspondingly, “substantially half” means nearly 50%, but permits slight variations because, especially with biosynthetically produced macromolecules, the most suitable case is never achieved, and certain deviations have always to be taken into consideration as enzymes do not work perfectly and catalysis usually involves a certain rate of errors. In the case of natural heparin, however, there is a strictly alternating sequence of N-acetylglucosamine and uronic acid units.

Furthermore, a process for the hemocompatible coating of surfaces intended for direct blood contact is disclosed. In said process, a natural and/or artificial surface is provided, and the oligosaccharides and/or polysaccharides described above are immobilized on said surface.

The immobilization of the oligosaccharides and/or polysaccharides on said surfaces can be effected by means of hydrophobic interactions, van der Waals' forces, electrostatic interactions, hydrogen bridges, ionic interactions, cross-linking of the oligosaccharides and/or polysaccharides and/or by covalent bonding to the surface. Preferred is the covalent linkage of the oligosaccharides and/or polysaccharides, more preferred the covalent individual point linkage (side-on bonding), and especially preferred the covalent end point linkage (end-on bonding).

Under “substantially the remaining sugar building units” is to be understood that 93% of the remaining sugar building units, preferred 96% and especially preferred 98% of the remaining 60% to 40% of the sugar building units have a carboxyl group.

Thus, stents are preferred which have as most lower layer a hemocompatible layer of the above mentioned heparin derivatives, chitosan derivatives and/or oligo- or polypeptides. On this layer rapamycin is present as pure active agent layer and/or in an embedded form in a matrix of a carrier substance.

### Polysulfones as Biostable Polymeric Carriers for Rapamycin

Surprisingly, it was found that for the coating of stents which are preferably in permanent contact with blood polysulfone, polyethersulfone and/or polyphenylsulfone and their derivatives are an extremely well suitable biocompatible and hemocompatible carrier for rapamycin.

A preferred thermoplastic polysulfone is synthesized from bisphenol A and 4,4′-dichlorophenylsulfone via polycondensation reactions (see following formula (II)).

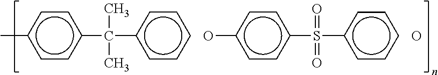

### Poly[oxy-1,4-phenylene-sulfonyl-1,4-phenylene-oxy-(4,4′-isopropylidenediphenylene)]

The polysulfones which are applicable for the coating according to the invention have the following general structure according to formula (I):

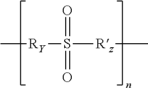

wherein

n represents the grade of polymerization, which is in the range from n=10 to n=10,000, preferably in the range from n=20 to n=3,000, further preferably in the range from n=40 to n=1,000, further preferably in the range from n=60 to n=500, further preferably in the range from n=80 to n=250 and particularly preferable in the range from n=100 to n=200.

Further, it is preferred if n is in such a range that a weight average of the polymer of 60,000-120,000 g/mol, preferably 70,000 to 99,000 g/mol, further preferably 80,000-97,000 g/mol, still more preferably 84,000-95,000 g/mol, and especially preferred 86,000-93,000 g/mol results.

Moreover, it is preferred if n is in such a range that the number average of the polymer in a range from 20,000-70,000 g/mol, preferably from 30,000-65,000 g/mol, further preferably 32,000-60,000, still more preferred 35,000-59,000, and particularly preferable from 45,000-58,000 g/mol results.

y and z are integer numbers in the range from 1 to 10, and R and R′ mean independently of each other an alkylene group having 1 to 12 carbon atoms, an aromatic group having 6 to 20 carbon atoms, a heteroaromatic group having 2 to 10 carbon atoms, a cycloalkylene group having 3 to 15 carbon atoms, an alkylenearylene group having 6 to 20 carbon atoms, an arylenealkylene group having 6 to 20 carbon atoms, an alkyleneoxy group having 1 to 12 carbon atoms, an aryleneoxygroup having 6 to 20 carbon atoms, a heteroaryleneoxy group having 6 to 20 carbon atoms, a cycloalkyleneoxy group having 3 to 15 carbon atoms, an alkylenearyleneoxy group having 6 to 20 carbon atoms or an arylenealkyleneoxy group having 6 to 20 carbon atoms. The above mentioned groups can have further substituents, particularly those which are described below by “substituted” polysulfones.

Examples for the groups R and R′ are —R¹—, —R²—, —R³—, —R⁴—, —R⁵—, —R⁶—, —R¹—R²—, —R³—R⁴—, —R⁵—R⁶—, —R¹—R²—R³—, —R⁴—R⁵—R⁶—, —R¹—R²—R³—R⁴—, —R¹—R²—R³—R⁴—R⁵— as well as —R¹—R²—R³—R⁴—R⁵—R⁶—;

wherein R¹, R², R³, R⁴, R⁵ and R⁶ represent independently of each other the following groups:
—CH₂—, —C₂H₄—, —CH(OH)—, —CH(SH)—, —CH(NH₂)—, —CH(OCH₃)—, —C(OCH₃)₂—, —CH(SCH₃)—, —C(SCH₃)₂—, —CH(NH(CH₃))—, —C(N(CH₃)₂)—, —CH(OC₂H₅)—, —C(OC₂H₅)₂—, —CHF—, —CHCl—, —CHBr—, —CF₂—, —CCl₂—, —CBr₂—, —CH (COOH)—, —CH(COOCH₃)—, —CH (COOC₂H₅)—, —CH(COCH₃)—, —CH(COC₂H₅)—, —CH(CH₃)—, —C(CH₃)₂—, —CH(C₂H₅)—, —C(C₂H₅)₂—, —CH(CONH₂)—, —CH(CONH(CH₃))—, —CH(CON(CH₃)₂)—, —C₃H₆—, —C₄H₈—, —C₆H₁₀—, cyclo-C₃H₄—, cyclo-C₃H₄—, cyclo-C₄H₆—, cyclo-C₅H₈—, —OCH₂—, —OC₂H₄—, —OC₃H₆—, —OC₄H₈—, —OC₅H₉—, —OC₆H₁₀—, —CH₂O—, —C₂H₄O—, —C₃H₆O—, —O₄H₈O—, —O₅H₉O—, —C₆H₁₀O—, —NHCH₂—, —NHC₂H₄—, —NHC₃H₆—, —NHC₄H₈—, —NHC₅H₉—, —NHC₆H₁₀—, —CH₂NH—, —C₂H₄NH—, —C₃H₆NH—, —C₄H₈NH—, —O₅H₉NH—, —O₆H₁₀NH—, —SOH₂—, —SC₂H₄—, —SC₃H₆—, —SC₄H₈—, —SC₅H₉—, —SC₆H₁₀—, —CH₂S—, —C₂H₄S—, —C₃H₆S—, —C₄H₈S—, —O₅H₉S—, —C₆H₁₀S—, —O₆H₄—, —O₆H₃(OH₃)—, —O₆H₃(O₂H₅)—, —O₆H₃(OH)—, —O₆H₃(NH₂)—, —C₆H₃(Cl)—, —C₆H₃(F)—, —C₆H₃(Br)—, —C₆H₃(OCH₃)—, —C₆H₃(SCH₃)—, —C₆H₃(COCH₃)—, —C₆H₃(COC₂H₅)—, —C₆H₃(COOH)—, —C₆H₃(COOCH₃)—, —C₆H₃(COOC₂H₅)—, —C₆H₃(NH(CH₃))—, —C₆H₃(N(CH₃)₂)—, —C₆H₃(CONH₂)—, —C₆H₃(CONH(CH₃))—, —C₆H₃(CON(CH₃)₂)—, —OC₆H₄—, —OC₆H₃(OH₃)—, —OC₆H₃(O₂H₅)—, —OC₆H₃(OH)—, —OC₆H₃(NH₂)—, —OC₆H₃(Cl)—, —OC₆H₃(F)—, —OC₆H₃(Br)—, —OC₆H₃(OCH₃)—, —OC₆H₃(SCH₃)—, —OC₆H₃(COCH₃)—, —OC₆H₃(COC₂H₅)—, —OC₆H₃(COOH)—, —OC₆H₃(COOCH₃)—, —OC₆H₃(COOC₂H₅)—, —OC₆H₃(NH(OH₃))—, —OC₆H₃(N(CH₃)₂)—, —OC₆H₃(CONH₂)—, —OC₆H₃(CONH(CH₃))—, —OC₆H₃(CON(CH₃)₂)—, —C₆H₄O—, —C₆H₃(OH₃)O—, —C₆H₃(C₂H₅)O—, —C₆H₃(OH)O—, —C₆H₃(NH₂)O—, —C₆H₃(Cl)O—, —C₆H₃(F)O—, —C₆H₃(Br)O—, —C₆H₃(OCH₃)O—, —C₆H₃(SCH₃)O—, —C₆H₃(COCH₃)O—, —C₆H₃(COC₂H₅)O—, —C₆H₃(COOH)O—, —C₆H₃(COOCH₃)O—, —C₆H₃(COOC₂H₅)O—, —C₆H₃(NH(CH₃))O—, —C₆H₃(N(CH₃)₂)O—, —C₆H₃(CON H₂)O—, —C₆H₃(CONH(CH₃))O—, —C₆H₃(CON(CH₃)₂)O—, —SC₆H₄—, —SC₆H₃(CH₃)—, —SC₆H₃(C₂H₅)—, —SC₆H₃(OH)—, —SC₆H₃(NH₂)—, —SC₆H₃(Cl)—, —SC₆H₃(F)—, —SC₆H₃(Br)—, —SC₆H₃(OCH₃)—, —SC₆H₃(SCH₃)—, —SC₆H₃(COCH₃)—, —SC₆H₃(COC₂H₅)—, —SC₆H₃(COOH)—, —SC₆H₃(COOCH₃)—, —SC₆H₃(COOC₂H₅)—, —SC₆H₃(NH(CH₃))—, —SC₆H₃(N(CH₃)₂)—, —SC₆H₃(CONH₂)—, —SC₆H₃(CONH(CH₃))—, —SC₆H₃(CON(CH₃)₂)—, —C₆H₄S—, —C₆H₃(CH₃)S—, —C₆H₃(C₂H₅)S—, —C₆H₃(OH)S—, —C₆H₃(NH₂)S—, —C₆H₃(Cl)S—, —C₆H₃(F)S—, —C₆H₃(Br)S—, —C₆H₃(OCH₃)S—, —C₆H₃(SCH₃)S—, —C₆H₃(COCH₃)S—, —C₆H₃(COC₂H₅)S—, —C₆H₃(COOH)S—, —C₆H₃(COOCH₃)S—, —C₆H₃(COOC₂H₅)S—, —C₆H₃(NH(CH)S—, —C₆H₃(N (CH₃)₂)S—, —C₆H₃(CONH)S—, —C₆H₃(CONH(CH₃))S—, —C₆H₃(CON (CH₃)₂)S—, —NH—C₆H₄—, —NH—C₆H₃(CH₃)—, —NH—C₆H₃(C₂H₅)—, —NH—C₆H₃(OH)—, —NH—C₆H₃(NH₂)—, —NH—C₆H₃(Cl)—, —NH—C₆H₃(F)—, —NH—C₆H₃(Br)—, —NH—C₆H₃(OCH₃)—, —NH—C₆H₃(SCH₃)—, —NH—C₆H₃(COCH₃)—, —NH—C₆H₃(COC₂H₅)—, —NH—C₆H₃(COOH)—, —NH—C₆H₃(COOCH₃)—, —NH—C₆H₃(COOC₂H₅)—, —NH—C₆H₃(NH(CH₃))—, —NH—C₆H₃(N(CH₃)₂)—, —NH—C₆H₃(CONH₂)—, —NH—C₆H₃(CONH(CH₃))—, —NH—C₆H₃(CON(CH₃)₂)—, —C₆H₄—NH—, —C₆H₃(CH₃)—NH—, —C₆H₃(C₂H₅)—NH—, —C₆H₃(OH)—NH—, —C₆H₃(NH₂)—NH—, —C₆H₃(Cl)—NH—, —C₆H₃(F)—NH—, —C₆H₃(Br)—NH—, —C₆H₃(OCH₃)—NH—, —C₆H₃(SCH₃)—NH—, —C₆H₃(COCH₃)—NH—, —C₆H₃(COC₂H₅)—NH—, —C₆H₃(COOH)—NH—, —C₆H₃(COOCH₃)—NH—, —C₆H₃(COOC₂H₅)—NH—, —C₆H₃(NH(CH₃))—NH—, —C₆H₃(N(CH₃)₂)—NH—, —C₆H₃(CONH₂)—NH—, —C₆H₃(CONH(CH₃))—NH—, —C₆H₃(CON(CH₃)₂)—NH—.

Especially preferred are polysulfones as well as their mixtures, wherein the groups —R¹—, —R²—, —R³—, —R¹—R²—, —R¹—R²—R³— represent independently of each other the following groups: —C₆H₄O—, —C(CH₃)₂—, —C₆H₄—, —C₆H₄SO₂—, —SO₂C₆H₄—, —OC₆H₄—, and —C₆H₄O—C(CH₃)₂—C₆H₄—.

R and R′ can further represent independently of each other preferably a moiety which is bound to the sulfone group in the formulas (II) to (XV).

According to the invention, the polysulfone or the polysulfones, respectively, for the biostable layer or the biostable layers are selected from the group which comprises: polyethersulfone, substituted polyethersulfone, polyphenylsulfone, substituted polyphenylsulfone, polysulfone block copolymers, perfluorinated polysulfone block copolymers, semifluorinated polysulfone block copolymers, substituted polysulfone block copolymers and/or mixtures of the above mentioned polymers.

The term “substituted” polysulfones is to be understood as polysulfones which have functional groups. Especially the methylene units can have one or two substituents and the phenylene units can have one, two, three, or four substituents. Examples for these substituents (also referred to as: X, X′, X″, X′″) are: —OH, —OCH₃, —OC₂H₅, —SH, —SCH₃, —SC₂H₅, —NO₂, —F, —Cl, —Br, —I, —N₃, —ON, —OCN, —NCO, —SCN, —NCS, —CHO, —COCH₃, —COC₂H₅, —COOH, —COON, —COOCH₃, —COOC₂H₅, —CONH₂, —CONHCH₃, —CONHC₂H₅, —CON(CH₃)₂, —CON(C₂H₅)₂, —NH₂, —NHCH₃, —NHC₂H₅, —N(CH₃)₂, —N(C₂H₅)₂, —SOCH₃, —SOC₂H₅, —SO₂CH₃, —SO₂C₂H₅, —SO₃H, —SO₃C H₃, —SO₃C₂H₅, —OCF₃, —O—COOCH₃, —O—COOC₂H₅, —NH—CO—NH₂, —NH—CS—NH₂, —NH—C(═NH)—NH₂, —O—CO—NH₂, —NH—CO—OCH₃, —NH—CO—OC₂H₅, —CH₂F —CHF₂, —CF₃, —CH₂Cl—CHCl₂, —CCl₃, —CH₂Br —CHBr₂, —CBr₃, —CH₂l—CHl₂, —Cl₃, —CH₃, —C₂H₅, —C₃H₇, —CH(CH₃)₂, —C₄H₉, —CH₂—CH(CH₃)₂, —CH₂—COOH, —CH(CH₃)—C₂H₅, —C(CH₃)₃, —H. Further preferred substituents or functional groups are —CH₂—X and —C₂H₄—X.

The following general structural formulas represent preferred repeating units for polysulfones. Preferably, the polymers only consist of these repeating units. However, it is also possible that in one polymer other repeating units or blocks are present besides the shown repeating units. Preferred are:

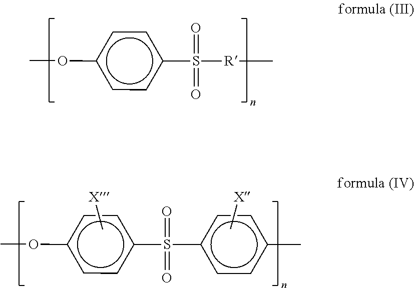

X, X′, n and R′ have independently of each other the above mentioned meaning.

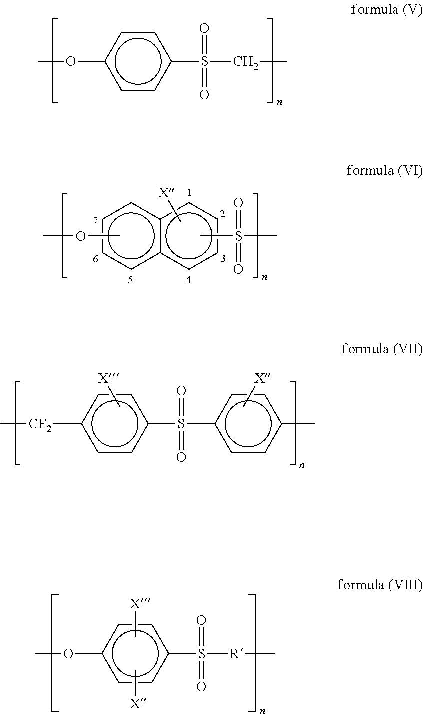

X, X′, n and R′ have independently of each other the above mentioned meaning.

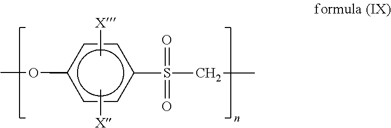

Further, polysulfones of the following general formula (X) are preferred:

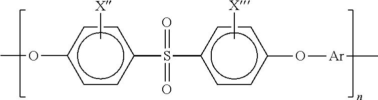

wherein Ar represents:

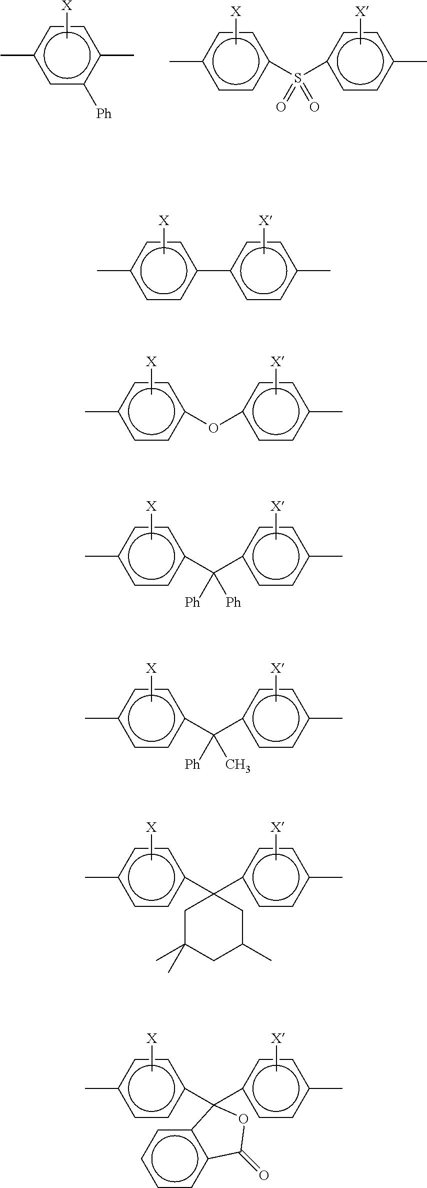

X, X′ and n have independently of each other the above mentioned meaning.

Furthermore, the following repeating units are preferred:

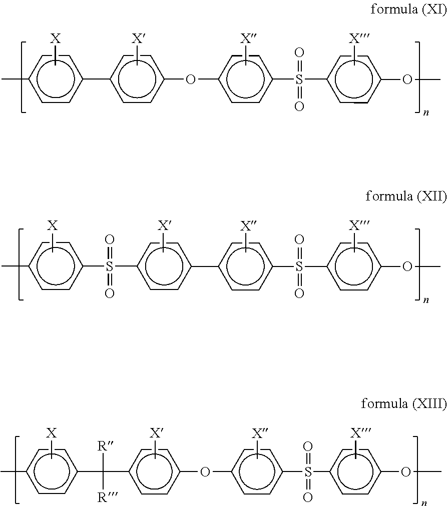

X, X′, X″, X′″ and n have independently of each other the above mentioned meaning. R″ and R′″ can represent independently of each other a substituent, as it is defined for X or X′, or can represent independently of each other a group —R¹—H or —R²—H.

Another preferred repeating unit has a cyclic substituent between two aromatic rings such as for example formula (XIV) or (XV):

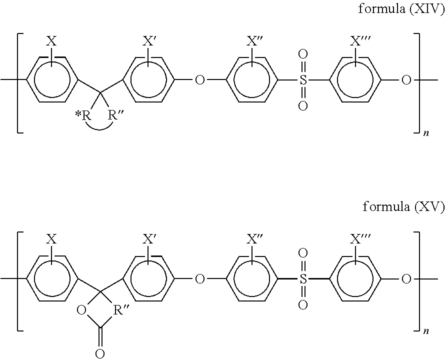

R″ preferably represents —CH₂—, —OCH₂—, —CH₂O—, —O—, —C₂H₄—, —C₃H₆—, —CH(OH)—. The group —*R—R″— preferably represents a cyclic ester, amide, carbonate, carbamate or urethane such as for example: —O—CO—O—, —O—CO—O—CH₂—, —O—CO—O—C₂H₄—, —CH₂—O—CO—O—CH₂—, —C₂H₄—, —C₃H₆—, —C₄H₈—, —O₅H₁₀—, —C₆H₁₂—, —O—CO—NH—, NH—CO—NH—, —O—CO—NH—CH₂—, —O—CO—NH—C₂H₄—, —NH—CO—NH—CH₂—, —NH—CO—NH—C₂H₄—, —NH—CO—O—CH₂—, —NH—CO—O—C₂H₄—, —CH₂—O—CO—NH—CH₂—, —C₂H₄—SO₂—, —C₃H₆—SO₂—, —C₄H₈—SO₂—, —C₂H₄—SO₂—CH₂—, —C₂H₄—SO₂—C₂H₄—, —C₂H₄—O—, —C₃H₆—O—, —C₄H₈—O—, —C₂H₄—O—CH₂—, —C₂H₄—O—C₂H₄—, —C₂H₄—CO—, —C₃H₆—CO—, —C₄H₈—CO—, —C₂H₄—CO—CH₂—, —C₂H₄—CO—C₂H₄—, —O—CO—CH₂—, —O—CO—C₂H₄—, —O—CO—C₂H₂—, —CH₂—O—CO—CH₂—, or cyclic esters, which contain an aromatic ring.

In the following, polymer analogous reactions will be described, which are known to a skilled person and serve for the modification of the polysulfones.

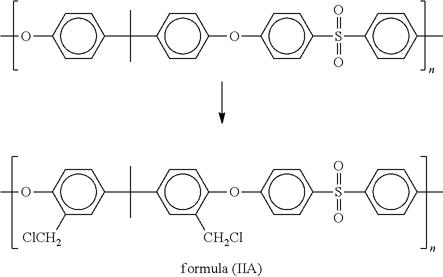

Chloromethylene groups as moieties X and X′ can be introduced by use of formaldehyde, ClSiMe₃ and a catalyst such as SnCl₄, which then can be further substituted. Via these reactions, for example hydroxyl groups, amino groups, carboxylate groups, ether or alkyl groups can be introduced by a nucleophilic substitution, which are bound to the aromat via a methylene group. A reaction with alcoholates, such as for example a phenolate, benzylate, methanolate, ethanolate, propanolate or isopropanolate results in a polymer in which a substitution occurred at over 75% of the chloromethylene groups. The following polysulfone with lipophilic side groups is obtained:

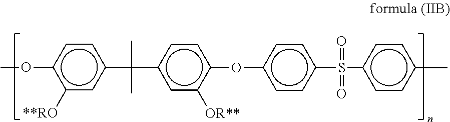

wherein

R** for example represents an alkyl moiety or aryl moiety.

The moieties X″ and X′″ can be introduced, as far as not yet present in the monomers, at the polymer by following reaction:

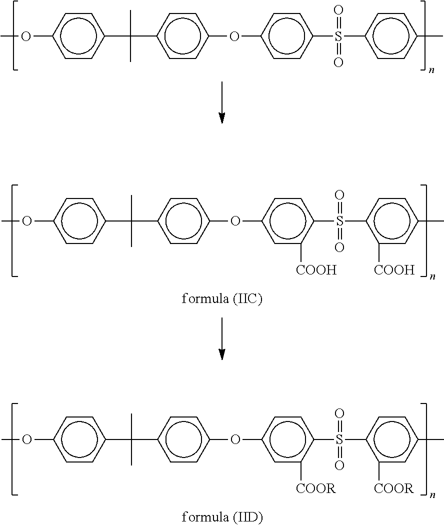

Besides an ester group, diverse other substituents can be introduced, by at first proceeding a single or double deprotonation by means of a strong base, e.g. n-BuLi or tert-BuLi, and by subsequently adding an electrophile. In the above exemplary case, carbon dioxide was added for the introduction of the ester group and the obtained carbonic acid group was esterified in another step.

A combination according to the invention of a polysulfone with lipophilic moieties and a polysulfone with lipophobic moieties is achieved for example by the use of polysulfone according to formula (IIB) together with polysulfone according to formula (IIC). The amount ratios of both polysulfones to each other can range from 98%:2% to 2%:98%. Preferred ratios are 10% to 90%, 15% to 85%, 22% to 78% and 27% to 73%, 36% to 64%, 43% to 57% and 50% to 50%. These percentage values are to be applied for any combination of hydrophilic and hydrophobic polysulfones and are not limited to the above-mentioned mixture.

An example of a polysulfone with hydrophilic and hydrophobic moieties in one molecule can be obtained for example by esterifying only incompletely the polysulfone according to formula (IIC) and thus, hydrophilic carboxylate groups and hydrophobic ester groups are present in one molecule. The mole ratio (number) of carboxylate groups to ester groups can be 5%:95% to 95%:5%. These percentage values are to be applied for any combination of hydrophilic and hydrophobic groups and are not limited to the aforementioned ones.

It is supposed that by means of this combination according to the invention of hydrophilic groups or, respectively, polymers with hydrophobic groups or, respectively, polymers, amorphous polymer layers are built on the medical product. It is very important that the polymer layers made of polysulfone are not crystalline or principally crystalline, as crystallinity results in rigid layers, which break and detach. Flexible polysulfone coatings serving as a barrier layer can be achieved only with amorphous or principally amorphous polysulfone layers.

Of course, it is also possible to apply monomers which are already substituted correspondingly for obtaining the desired substitution pattern after the polymerization being effected. The corresponding polymers then result by the known way according to the following reaction scheme:

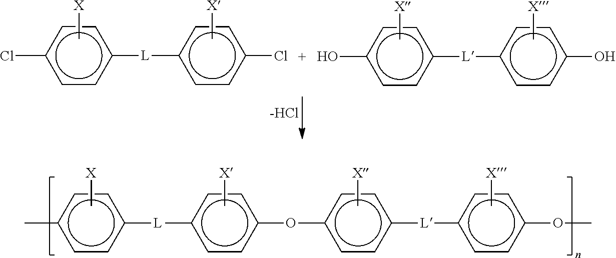

wherein

L and L′ represent for example the following groups independently of each other: —SO₂—, —C(CH₃)₂—, —C(Ph)₂- or —O—. L and L′ can thus have the meanings of the corresponding groups in the formulas (I) to (XV). Such nucleophilic substitution reactions are known to the one skilled in the art, which are illustrated exemplarily by the above scheme.

As already mentioned, it is especially preferred if the polymers have hydrophilic and hydrophobic properties, on the one hand within one polymer and on the other hand by use of at least one hydrophilic polymer in combination with at least one hydrophobic polymer. Thus, it is preferred if for example X and X′ have hydrophilic substituents and X″ and X′″ have hydrophobic substituents, or vice versa.

As hydrophilic substituents can be applied: —OH, —CHO, —COOH, —COO⁻, —CONH₂, —NH₂, —N⁺(CH₃)₄, —NHCH₃, —SO₃H, —SO₃⁻, —NH—CO—NH₂, —NH—CS—NH₂, —NH—C(═NH)—NH₂, —O—CO—NH₂ and especially protonated amino groups.

As hydrophobic substituents can be applied: —H, —OCH₃, —OC₂H₅, —SCH₃, —SC₂H₅, —NO₂, —F, —Cl, —Br, —I, —N₃, —ON, —OCN, —NCO, —SON, —NCS, —COCH₃, —COC₂H₅, —COCN, —COOCH₃, —COOC₂H₅, —CONHC₂H₅, —CON(CH₃)₂, —CON(C₂H₅)₂, —NHC₂H₅, —N(CH₃)₂, —N(C₂H₅)₂, —SOCH₃, —SOC₂H₅, —SO₂CH₃, —SO₂C₂H₅, —SO₃CH₃, —SO₃C₂H₅, —OCF₃, —O—COOCH₃, —O—COOC₂H₅, —NH—CO—OCH₃, —NH—CO—OC₂H₅, —CH₂F—CHF₂, —CF₃, —CH₂Cl—CHCl₂, —CCl₃, —CH₂Br —CHBr₂, —CBr₃, —CH₂l—CHl₂, —Cl₃, —CH₃, —C₂H₅, —C₃H₇, —CH(CH₃)₂, —C₄H₉, —CH₂—CH(CH₃)₂, —CH₂—COOH, —CH(CH₃)—C₂H₅, —C(CH₃)₃.

Moreover, cyclic polysulfones are preferred, which have for example a structure as shown in formula (XVI):

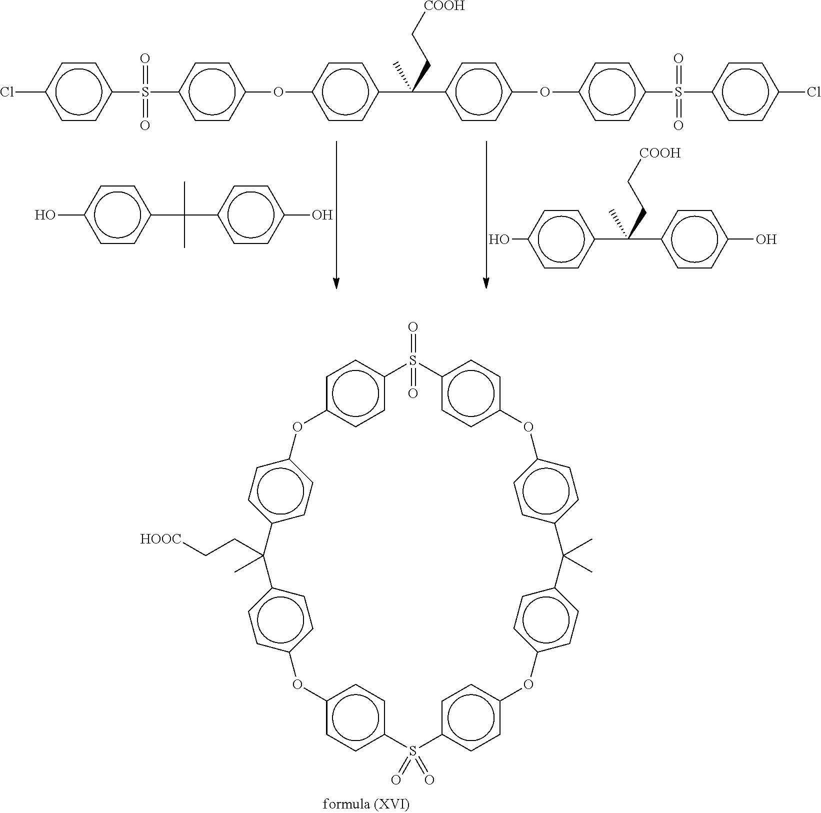

The carboxyethylene group is not essential for the above exemplary reaction. Instead of the carboxyethylene and the methyl substituents, any other substituents or also hydrogen can be present.

### Oils and Fats as Carrier Substances

Besides the above mentioned biostable and biodegradable polymers as carrier matrix for rapamycin and other active agents also physiologically acceptable oils, fats, lipids, lipoids and waxes can be used.

As such oils, fats and waxes which can be used as carrier substances for rapamycin or other active agents or as active agent-free layers, especially to players, substances are suitable which can be represented by the following general formulas:

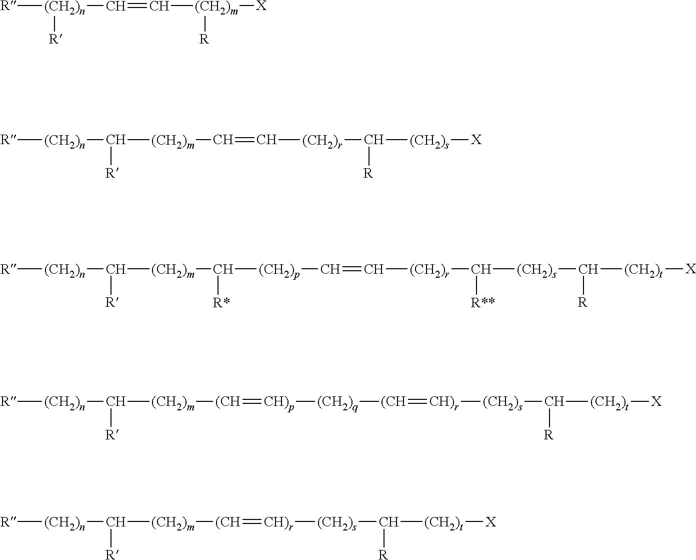

wherein

R, R′, R″, R* and R** are independently of each other alkyl, alkenyl, alkinyl, heteroalkyl, cycloalkyl, heterocyclyl groups having 1 to 20 carbon atoms, aryl, arylalkyl, alkylaryl, heteroaryl groups having 3 to 20 carbon atoms or functional groups and preferably represent the following groups: —H, —OH, —OCH₃, —OC₂H₅, —OC₃H₇, —O-cyclo-C₃H₅, —OCH(OH₃)₂, —OC(OH₃)₃, —OC₄H₉, —OPh, —OCH₂-Ph, —OCPh₃, —SH, —SCH₃, —SC₂H₅, —NO₂, —F, —Cl, —Br, —I, —CN, —OCN, —NCO, —SON, —NOS, —CHO, —COCH₃, —COC₂H₅, —COC₃H₇, —CO-cyclo-C₃H₅, —COCH(CH₃)₂, —OC(CH₃)₃, —COOH, —COOCH₃, —COOC₂H₅, —COOC₃H₇, —COO-cyclo-C₃H₅, —COOCH(CH₃)₂, —COOCH(CH₃)₃, —OOC—CH₃, —OOC—C₂H₅, —OOC—O₃H₇, —OOC-cyclo-C₃H₅, —OOC—CH(CH₃)₂, —OOC—O(CH₃)₃, —CONH₂, —CONHCH₃, —CONHC₂H₅, —CONHC₃H₇, —CON(CH₃)₂, —CON(C₂H₅)₂, —CON(C₃H₇)₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-O₃H₅)₂, —N[CH(OH₃)₂]₂, —N[C(OH₃)₃]₂, —SOCH₃, —SOC₂H₅, —SOC₃H₇, —SO₂OH₃, —SO₂O₂H₅, —SO₂O₃H₇, —SO₃H, —SO₃OH₃, —SO₃O₂H₅, —SO₃O₃H₇, —OCF₃, —OC₂F₅—O—COOCH₃, —O—COOC₂H₅, —O—COOC₃H₇, —O—COO-cyclo-C₃H₅, —O—COOCH(CH₃)₂, —O—COOC(OH₃)₃, —NH—CO—NH₂, —NH—CO—NHCH₃, —NH—CO—NHC₂H₅, —NH—CO—N(CH₃)₂, —NH—CO—N(C₂H₅)₂, —O—CO—NH₂, —O—CO—NHCH₃, —O—CO—NHC₂H₅, —O—CO—NHC₃H₇, —O—CO—N(CH₃)₂, —O—CO—N(C₂H₅)₂, —O—CO—COH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F, —CHF₂, —CF₃, —CH₂Cl, —CH₂Br, —CH₂I, —CH₂—CH₂F, —CH₂—CHF₂, —CH₂—CF₃, —CH₂—OH₂Cl, —CH₂—CH₂Br₃—CH₂—OH₂I, —CH₃, —O₂H₅, —O₃H₇, -cyclo-C₃H₅, —CH(CH₃)₂, —C(CH₃)₃, —O₄H₉, —CH₂—CH(CH₃)₂, —CH(CH₃)—O₂H₅, -Ph, —CH₂-Ph, —CPh₃, —CH═CH₂, —CH₂—CH═CH₂, —C(CH₃)═CH₂, —CH═CH—CH₃, —O₂H₄—CH═CH₂, —CH═C(CH₃)₂, —C≡CH, —C≡C—CH₃, —CH₂—C≡CH;

X is an ester group or amide group and especially —O-alkyl, —O—CO-alykl, —O—CO—O-alkyl, —O—CO—NH-alkyl, —O—CO—N-dialkyl, —CO—NH-alkyl, —CO—N-dialkyl, —CO—O-alkyl, —CO—OH, —OH;
m, n, p, q, r, s and t are independently of each other integers from 0 to 20, preferred from 0 to 10.

The term “alkyl” for example in —CO—O-alkyl is preferably one of the alkyl groups mentioned for the aforesaid groups R, R′ etc., such as —CH₂-Ph. The compounds of the aforesaid general formulas can be present also in the form of their salts as racemates or diastereomeric mixtures, as pure enantiomers or diastereomers as well as mixtures or oligomers or copolymers or block copolymers. Moreover, the aforesaid substances can be used in mixture with other substances such as biostable and biodegradable polymers and especially in mixture with the herein mentioned oils and/or fatty acids. Preferred are such mixtures and individual substances which are suitable for polymerization, especially for auto polymerization.

The substances suitable for the polymerization, especially autopolymerization, comprise i.a. oils, fats, fatty acids as well as fatty acid esters, which are described in more detail below. In the case of the lipids are preferably concerned mono- or poly-unsaturated fatty acids and/or mixtures of these unsaturated fatty acids in the form of their triglycerides and/or in non glycerin bound, free form.

Preferably the unsaturated fatty acids are chosen from the group, which comprises oleic acid, eicosapentaenoic, acid, timnodonic acid, docosahexaenoic acid, arachidonic acid, linoleic acid, α-linolenic acid, γ-linolenic acid as well as mixtures of the aforementioned fatty acids. These mixtures comprise especially mixtures of the pure unsaturated compounds.

As oils are preferably used linseed oil, hempseed oil, corn oil, walnut oil, rape oil, soy bean oil, sun flower oil, poppy-seed oil, safflower oil (Färberdistelol), wheat germ oil, safflor oil, grape-seed oil, evening primrose oil, borage oil, black cumin oil, algae oil, fish oil, cod-liver oil and/or mixtures of the aforementioned oils. Especially suitable are mixtures of the pure unsaturated compounds.

Fish oil and cod-liver oil mainly contain eicosapentaenoic acid (EPA C20:5) and docosahexaenoic acid (DHA C22:6) besides of little a-linolenic acid (ALA C18:3). In the case of all of the three fatty acids, omega-3 fatty acids are concerned, which are required in the organism as important biochemical constituting substance for numerous cell structures (DHA and EPA), for example as already mentioned, they are fundamental for the build up and continuance of the cell membrane (sphingolipids, ceramides, gangliosides). Omega-3 fatty acids can be found not only in fish oil, but also in vegetable oils. Further unsaturated fatty acids, such as the omega-6 fatty acids, are present in oils of herbal origin, which here partly constitute a higher proportion than in animal fats. Hence different vegetable oils such as linseed oil, walnut oil, flax oil, evening primrose oil with accordingly high content of essential fatty acids are recommended as especially high-quality and valuable edible oils. Especially linseed oil represents a valuable supplier of omega-3 and omega-6 fatty acids and is known for decades as high-quality edible oil.

As participating substances in the polymerization reaction the omega-3 as well as the omega-6 fatty acids are preferred as well as all of the substances, which have at least one omega-3 and/or omega-6 fatty acid moiety. Suchlike substances demonstrate also a good capability for autopolymerization. The ability of curing, i.e. the ability for autopolymerization, is based in the composition of the oils, also referred to as toweling oils, and goes back to the high content of essential fatty acids, more precisely to the double bonds of the unsaturated fatty acids. Exposed to air radicals are generated by means of the oxygen on the double bond sites of the fatty acid molecules, which initiate and propagate the radical polymerization, such that the fatty acids cross-link among themselves under loss of the double bonds. With the clearing of the double bond in the fat molecule the melting point increases and the cross linking of the fatty acid molecules causes an additional curing. A high molecular resin results, which covers the medical surface homogeneously as flexible polymer film.

The auto-polymerization is also referred to as self polymerization and can be initiated for example by oxygen, especially by aerial oxygen. This auto-polymerization can also be carried out under exclusion of light. Another possibility exists in the initiation of the auto-polymerization by electromagnetic radiation, especially by light. Still another but less preferred variant is represented by the auto-polymerization initiated by chemical decomposition reactions, especially by decomposition reactions of the substances to be polymerized.

The more multiple bonds are present in the fatty acid moiety, the higher is the degree of cross-linking. Thus, the higher the density of multiple bonds is in an alkyl moiety (fatty acid moiety) as well as in one molecule, the smaller is the amount of substances, which participate actively in the polymerization reaction.

The content of substances participating actively in the polymerization reaction in respect to the total amount of all of the substances deposited on the surface of the medical product is at least 25% by weight, preferred 35% by weight, more preferred 45% by weight and especially preferred 55% by weight.

The following table 1 shows a listing of the fatty acid constituents in different oils, which are preferably used in the present invention.

TABLE 1

| | | | | Eicosa- | Docosa- |
|---|---|---|---|---|---|
| | Oleic | Linoleic | Linolenic | pentaenoic | hexaenoic |
| | acid | acid | acid | acid | acid |
| | (C 18:1) | (C 18:2) | (C 18:3) | (C 20:5) | (C 22:6) |
| Oil species | omega-9 | omega-6 | omega-3 | omega-3 | omega-3 |
| Olive oil | 70 | 10 | 0 | 0 | 0 |
| Corn oil | 30 | 60 | 1 | 0 | 0 |
| Linseed oil | 20 | 20 | 60 | 0 | 0 |
| Cod-liver oil | 25 | 2 | 1 | 12 | 8 |
| Fish oil | 15 | 2 | 1 | 18 | 12 |

The oils and mixtures of the oils, respectively, used in the coating according to the invention contain an amount of unsaturated fatty acids of at least 40% by weight, preferred an amount of 50% by weight, more preferred an amount of 60% by weight, further preferred an amount of 70% by weight and especially preferred an amount of 75% by weight of unsaturated fatty acids. Should commercially available oils, fats or waxes be used, which contain a lower amount of compounds with at least one multiple bond than 40% by weight, so unsaturated compounds can be added in the quantity, that the amount of unsaturated compounds increases to over 40% by weight. In the case of an amount of less than 40% by weight the polymerization rate decreases too strong, so that homogeneous coatings cannot be guaranteed any more.

The property to polymerize empowers especially the lipids with high amounts of poly-unsaturated fatty acids as excellent substances for the present invention.

So the linoleic acid (octadecadienoic acid) has two double bonds and the linolenic acid (octadecatrienoic acid) has three double bonds. Eicosapentaenoic acid (EPA C20:5) has five double bonds and docosahexaenoic acid (DHA C22:6) has six double bonds in one molecule. With the number of double bonds also the readiness to the polymerization increases. These properties of the unsaturated fatty acids and of their mixtures as well as their tendency for auto-polymerization can be used for the biocompatible and flexible coating of medical surfaces especially of stents with e.g. fish oil, cod-liver oil or linseed oil (see examples 13-18).

Linoleic acid is also referred to as cis-9, cis-12-octadecadienoic acid (chemical nomenclature) or as Δ9,12-octadecadienoic acid or as octadecadienoic acid (18:2) and octadecadienoic acid 18:2 (n-6), respectively, (biochemical and physiological nomenclature, respectively). In the case of octadecadienoic acid 18:2 (n-6) n represents the number of carbon atoms and the number “6” indicates the position of the final double bond. Thus, 18:2 (n-6) is a fatty acid with 18 carbon atoms, two double bonds and with a distance of 6 carbon atoms from the final double bond to the external methyl group.

Preferably used are for the present invention the following unsaturated fatty acids as substances, which participate in the polymerization reaction and substances, respectively, which contain these fatty acids, or substances, which contain the alkyl moiety of these fatty acids, i.e. without the carboxylate group (—COOH).

TABLE 1
Monoolefinic fatty acids

| Systematic name | Trivial name | Short form |
|---|---|---|
| cis-9-tetradecenoic acid | myristoleic acid | 14:1(n-5) |
| cis-9-hexadecenoic acid | palmitoleic acid | 16:1(n-7) |
| cis-6-octadecenoic acid | petroselinic acid | 18:1(n-12) |
| cis-9-octadecenoic acid | oleic acid | 18:1(n-9) |
| cis-11-octadecenoic acid | vaccenic acid | 18:1(n-7) |
| cis-9-eicosenoic acid | gadoleinic acid | 20:1(n-11) |
| cis-11-eicosenoic acid | gondoinic acid | 20:1(n-9) |
| cis-13-docosenoic acid | erucinic acid | 22:1(n-9) |
| cis-15-tetracosenoic acid | nervonic acid | 24:1(n-9) |
| t9-octadecenoic acid | elaidinic acid | |
| t11-octadecenoic acid | t-vaccenic acid | |
| t3-hexadecenoic acid | | trans-16:1(n-13) |

TABLE 2
Poly-unsaturated fatty acids

| Systematic name | Trivial name | Short form |
|---|---|---|
| 9,12-octadecadienoic acid | linoleic acid | 18:2(n-6) |
| 6,9,12-octadecatrienoic acid | γ-linolenic acid | 18:3(n-6) |
| 8,11,14-eicosatrienoic acid | dihomo-γ- | 20:3(n-6) |
| | linolenic acid | |
| 5,8,11,14-eicosatetraenoic acid | arachidonic acid | 20:4(n-6) |
| 7,10,13,16-docosatetraenoic acid | — | 22:4(n-6) |
| 4,7,10,13,16-docosapentaenoic | — | 22:5(n-6) |
| acid | | |
| 9,12,15-octadecatrienoic acid | α-linolenic acid | 18:3(n-3) |
| 6,9,12,15-octadecatetraenoic | stearidonic acid | 18:4(n-3) |
| acid | | |
| 8,11,14,17-eicosatetraenoic acid | — | 20:4(n-3) |
| 5,8,11,14,17-eicosapentaenoic | EPA | 20:5(n-3) |
| acid | | |
| 7,10,13,16,19-docosapentaenoic | DPA | 22:5(n-3) |
| acid | | |
| 4,7,10,13,16,19-docosahexaenoic | DHA | 22:6(n-3) |
| acid | | |
| 5,8,11-eicosatrienoic acid | meadic acid | 20:3(n-9) |
| 9c,11t,13t-eleostearinoic acid | | |
| 8t,10t,12c-calendinoic acid | | |
| 9c,11t,13c-catalpicoic acid | | |
| 4,7,9,11,13,16,19-docosahepta- | stellaheptaenic | |
| decanoic acid | acid | |
| | taxolic acid | all-cis-5,9-18:2 |
| | pinolenic acid | all-cis-5,9,12- |
| | | 18:3 |
| | sciadonic acid | all-cis-5,11,14- |
| | | 20:3 |

TABLE 3
Acetylenic fatty acids

| Systematic name | Trivial name |
|---|---|
| 6-octadecynoic acid | taririnic acid |
| t11-octadecen-9-ynoic acid | santalbinic or ximeninic acid |
| 9-octadecynoic acid | stearolinic acid |
| 6-octadecen-9-ynoic acid | 6,9-octadeceninic acid |
| t10-heptadecen-8-ynoic acid | pyrulinic acid |
| 9-octadecen-12-ynoic acid | crepenynic acid |
| t7,t11-octadecadiene-9-noic acid | heisterinic acid |
| t8,t10-octadecadiene-12-ynoic acid | — |
| 5,8,11,14-eicosatetraynoic acid | ETYA |

After accomplishment of the described polymerization of the substances containing one linear or branched and one substituted or non-substituted alkyl moiety with at least one multiple bond, a surface of a medical product is obtained, which is at least partially provided with one polymer layer. In the ideal case a homogeneous continuously thick polymer layer is formed on the total external surface of the stent or a catheter balloon with or without a crimped stent. This polymer layer on the surface of the stent or the catheter balloon with or without stent consists of the substances participating in the polymerization reaction and includes the substances in the polymer matrix participating not actively in the polymerization reaction and/or active agents and/or rapamycin. Preferably the occlusion is adapted to allow the substances not participating in the polymerization, especially rapamycin and additional active agent, to diffuse out from the polymer matrix.

The biocompatible coating of the polymerized substances provides for the necessary blood compatibility of the stent or catheter balloon with or without stent and represents at the same time a suitable carrier for rapamycin and other active agents. An added active agent (or active agent combination), which is homogeneously distributed over the total surface of the stent and/or catheter balloon effects that the population of the surface by cells, especially by smooth muscle and endothelial cells, takes place in a controlled way. Thus, rapid population and overgrowth with cells on the stent surface does not take place, which could result in restenosis, however the population with cells on the stent surface is not completely prevented by a high concentration of a medicament, which involves the danger of a thrombosis. This combination of both effects awards the ability to the surface of a medical product according to the invention, especially to the surface of a stent, to grow rapidly into the vessel wall and reduces both the risk of restenosis and the risk of thrombosis. The release of the active agent or of the active agents spans over a period of 1 to 12 months, preferably 1 to 2 months after implantation.

Further preferred stents with rapamycin as active agent for elution offer a clearly increased surface for the loading with rapamycin as with these stents not only the stent struts but also the interstices between the stent struts are coated with a polymer or carrier matrix in which rapamycin is present. Such completely, i.e. stent struts and strut interstices, coated stents are manufactured according to a special method which is described in detail in the International patent application PCT/DE 2006/000766 having the title “Vollflächige Beschichtung von GefäBstützen” as well as in the German patent application DE 10 2005 021 622.6 of the Hemoteq GmbH.

This aim was achieved by completely covering the surface of the lattice-shaped or mesh-like scaffolding of the endoprosthesis. The term completely coating refers to a coating which entirely covers the interstices. Said coating can also be described as a continuous, i.e., a film is formed on an interstice, wherein said film only abuts the struts defining said interstice. Said coating extends over the interstice like a suspension bridge, which is only attached on its extremities and does not abut a solid ground in the interstice. For ensuring that this coating layer, which covers the entire surface, sufficiently adheres to the struts or respectively the endoprosthesis, the struts are being at least partially coated with a polymer A in a first coating step, the interstices, though, are not covered, and after wetting or respectively partially dissolving this first polymer coating layer, the step of completely coating the surface with a polymer B follows in a second coating step, wherein the first polymer coating layer conveys improved adhesion properties to the second polymer layer, which is supposed to be applied on the entire surface or respectively it is supposed to be a continuous layer.

Polymer A and polymer B can also be identical and advantageously they are different only as far as their concentration in the coating solution is concerned.

The struts or respectively the intersection points are enclosed by the first coating like a tube or an insulation around a wire; nevertheless this coating only surrounds the individual struts and does not yet interconnect two adjacent struts. The first coating serves as a support layer for imparting improved adhesion properties to the superjacent coating which is supposed to extend over the interstices between the struts and the intersection points.

Moreover, the individual struts or intersection points of the endoprosthesis may have recesses or cavities which, for example, could be filled with a pharmacological agent and be covered with the first polymer coating and the second coating. Such covering of such recesses and cavities is prior art and is to be considered as a preferred embodiment, but not as the principal aspect of the present invention.

The uncoated endoprosthesis or respectively the bare stent can be made of conventional materials such as medical stainless steel, titanium, chromium, vanadium, tungsten, molybdenum, gold, nitinol, magnesium, zinc, alloys of the aforementioned metals, or can be composed of ceramic materials or polymers. These materials are either self-expandable or balloon-expandable and biostable or biodegradable.

Preferably, the coating step b) is performed by means of spray coating or electrospinning, whereas the steps c) and d) are preferably performed by means of dip coating, micropipetting, electrospinning and/or the “soap bubble method”.

The polymer surface can be coated in a further step completely or partially with a polymer C on the inner surface and/or on the outer surface. Thus, it is important, for example for the luminal side of a tracheobronchial stent that it remains sufficiently lubricious for not interfering with the evacuation of secretion, mucus, and the like. The hydrophilicity can be increased by coating with an appropriate polymer such as polyvinylpyrrolidone (PVP).

This coating method overcomes the described shortcomings of the prior art with respect to complete surface coating and thus, eliminates the risks which the patient is exposed to.

Such medical devices which can be used according to the invention can be coated, on the one hand, by applying a coating on the solid material, for example the individual struts of a stent, and by filling the open area which is defined by the struts with a polymer layer B. This polymer layer is capable of covering the interstices of the stent struts coated with polymer A thanks to the polymer properties. The stability of the coat is a function of the two combined layers of polymer B and polymer A, which enclose the elements of the medical device. Thus any medical device having such interstices in the surface structure can be coated in accordance with the invention, as is the case for example with stents showing such interstices between the individual struts.

A biodegradable and/or biostable polymer A for the first coating and of a biodegradable or reabsorbable polymer B and/or biostable polymer for the covering second coating depending on the type of application may be used.

Furthermore, in a step prior to the step of coating with polymer A, a hemocompatible layer preferably can be bound covalently to the uncoated surface of the medical device or can be immobilized on the same by means of cross-linking, for example with glutardialdehyde. Such layer which does not activate the blood coagulation is useful when uncoated stent material can come into contact with blood. Thus, it is preferred firstly to provide a partially coated stent, such as for example described in US 595,159 for the treatment of aneurysms, with such hemocompatible layer.

Furthermore, it is preferred that the outer surface resulting from the second step of completely coating the surface be not even or plane but that the structure of a stent i.e. the structure of the struts, be still visible. The advantage thereof consists in the fact that the outer coated surface of the endoprosthesis facing the vessel wall has a corrugated and rough structure, which assures an improved fixation within the vessel.

Polymer A which surrounds the stent struts can contain an additional antiproliferative, antimigrative, antiangiogenic, anti-inflammatory, antiphlogistic, cytostatic, cytotoxic and/or antithrombotic active agent, wherein polymer B which covers the stents completely contains the active agent rapamycin. Thus, the rapamycin-eluting surface is clearly increased in comparison to a conventional coating which only surrounds the individual stent struts (see example No. 18).

The concentration of rapamycin and of other active agent if present is preferably in the range of 0.001-500 mg per cm² of the completely coated surface of the endoprosthesis, i.e. the surface is calculated taking into consideration the total surface of the coated struts and the surface of the covered interstices between the struts.

The methods according to the invention are adapted for coating for example endoprostheses and in particular stents such as for example coronary stents, vascular stents, tracheal stents, bronchial stents, urethral stents, esophageal stents, biliary stents, renal stents, stents for use in the small intestine, stents for use in the large intestine. Moreover, guiding wires, helices, cathethers, canulas, tubes as well as generally tubular implants or parts of the above mentioned medical devices can be coated according to the invention provided that a structural element comparable to a stent is contained in such medical device. As far as expandable medical devices or respectively endoprostheses are used, the coating preferably is carried out during the expanded state of the respective device.

The coated medical devices are preferably used for maintaining patency of any tubular structure, for example the urinary tract, esophaguses, tracheae, the biliary tract, the renal tract, blood vessels in the whole body including brain, duodenum, pilorus, the small and the large intestine, but also for maintaining the patency of artificial openings such as used for the colon or the trachea.

Thus, the coated medical devices are useful for preventing, reducing or treating stenoses, restenoses, arterioscleroses, atheroscleroses and any other type of vessel occlusion or vessel obstruction of lumens or openings.

Furthermore, it is preferred that the length of the complete coating layer which contains polymer B exceeds the length of the endoprosthesis and does not correspond to the end of the endoprothesis. In a further step, the overlapping part of the shell is placed around the edges of the endoprosthesis on the outer surface and the thus formed edges are being integrated into the subjacent polymer layer B under pressure and increased temperature. Thus, a continuous coating also of the edges of the endoprosthesis is assured, which eliminates at the same time the danger of detachment on these weak points. Moreover, a handling element can be mounted below the edge by means of which the stent can be removed safely at any time. Thus, a polymer fiber can be disposed circumferentially in the folding, wherein the fiber projects through the polymer layer from the edge to the outer surface in the form of a loop on one or two opposite sides.

Another possibility consists in the use of this marginal region as a reservoir for active agents or respectively for introducing active agents especially into this marginal region, wherein these active agents can be different from those possibly present in/on the completely coated surface of the hollow body.

Therein, the shell enclosing the stent is provided with the flexibility of the stent, but also contributes in imparting mechanical stiffness to the medical device. Additionally, there exists the possibility of introducing active agents in a side-specific manner, such as a cytostatic which can diffuse from the outer surface into the vessel wall, and for example an antibiotic which prevents infections on the inner surface of the medical device. Moreover, further optimizations concerning the adaptation to the physiological conditions at the respective implantation site can be achieved thanks to the possibility of applying different coatings on the inner and outer surfaces.

Further additives are possible, e.g. substances such as barium sulfate or precious metals, which allow for imaging an implanted, thus coated medical device in radiograms. Furthermore, the outer surface and the inner surface can be enclosed with different materials, such as described above. Thus, for example, a medical device which has a hydrophobic polymer shell on the outer surface whereas the inner surface is made of hydrophilic polymer can be manufactured.

This method offers a variety of possibilities for applying any biostable or biodegradable coating materials containing or not containing additives on medical devices, if necessary in the form of a shell.

At the same time, the coating can add to the mechanical stiffness of an implant without affecting the flexibility thereof.

Thus, up to now, e.g. the use of stents for the restriction of biliary tract carcinomas is not a standard procedure. However, in only 10% of the cases, a surgical removal is successful. Medium life expectancy of such patients is of 1 year. The use of an implant completely coated according to this method and adapted to application in the biliary tract, which could optionally contain a chemotherapeutic agent, could on the one hand prevent the constriction of the body lumen in that the endoprosthesis exerts a certain counter pressure and at the same time, could slow down or even stop tumor growth and thus would at least provide a life prolonging treatment while maintaining high or good quality of life (example 18).

Furthermore, the coating according to the invention can also be used in the vascular system. In the case of the formation of aneurysms it can be used for example in a manner that prevents an increase of the aneurysm due to the continued supply with blood (example 19).

Further embodiments according to the invention for increasing the surface refer to catheter systems, especially dilatation catheter systems, comprising a catheter balloon with a crimped stent. In these systems an uncoated or coated stent is crimped to the catheter balloon and then coated together with the catheter balloon. The coating can be carried out in a way that the free interstices between the individual stent struts of the crimped stent serve as reservoirs for an active agent or rapamycin. For example, rapamycin or one of the active agents mentioned herein can be dissolved in a suitable solvent and applied to the stent or balloon. Active agent and solvent flow into the interstices between the individual stent struts and into the interstices between catheter balloon and inner side of the stent, wherein the solvent evaporates and the pure active agent remains. Then, one or more carrier layers can be applied to the catheter balloon having the stent.

A preferred variant of this embodiment has pure paclitaxel between the stent struts and between balloon and stent that was applied by spraying or dipping method and remains there after evaporation of the solvent. This first paclitaxel coating is then covered by a preferably biodegradable polymer and/or preferably polar, hydrophilic polymer which contains the active agent rapamycin.

Another preferred embodiment has no carrier or no polymer layer but only pure rapamycin which was applied together with a solvent to the stent and catheter balloon and remains after evaporation of the solvent on the stent and balloon.

A third preferred embodiment comprises a stent which is coated with a preferably biostable polymer containing rapamycin and crimped to the balloon. The uncoated catheter balloon with rapamycin-containing coated stent is then sprayed with paclitaxel in a suitable solvent such that after evaporation of the solvent an irregular layer of pure paclitaxel is present on the stent and balloon.

### Contrast Agents

Of special interest are those embodiments according to the invention which use as matrix or carrier for rapamycin no polymers but low-molecular chemical compounds and especially contrast agents and contrast agent analogues.

Suchlike contrast agents and/or contrast agent analogues mostly contain barium, iodine, manganese, iron, lanthanum, cerium, praseodymium, neodymium, samarium, europium, gadolinium, terbium, dysprosium, holmium, erbium, thulium, ytterbium and/or lutetium preferably as ions in the bound and/or complex form.

In principle, contrast agents are to be distinguished for different imaging methods. On the one hand, there are contrast agents which are used in x-ray examinations (x-ray contrast agents) or contrast agents which are used in magnetic resonance tomography examinations (MR contrast agents).

In the case of x-ray contrast agents substances are concerned which result in an increased absorption of penetrating x-rays with respect to the surrounding structure (so-called positive contrast agents) or which let pass penetrating x-rays unhindered (so-called negative contrast agents).

Preferred x-ray contrast agents are those which are used for imaging of joints (arthrography) and in CT (computer tomography). The computer tomograph is a device for generating sectional images of the human body by means of x-rays.

Although according to the invention also x-rays can be used for the detection in the imaging methods this radiation is not preferred due to its harmfulness. It is preferred when the penetrating radiation is not an ionizing radiation.

As imaging methods are used x-ray images, computer tomography (CT), nuclear spin tomography, magnetic resonance tomography (MRT) and ultrasound, wherein nuclear spin tomography and magnetic resonance tomography (MRT) are preferred.

Thus, as substances which due to their ability of being excited by penetrating radiation allow for the detection of the medical device in in-vivo events by imaging methods are especially those contrast agents preferred which are used in computer tomography (CT), nuclear spin tomography, magnetic resonance tomography (MRT) or ultrasound. The contrast agents used in MRT are based on the mechanism of action that they effect a change of the magnetic behavior of the structures to be differentiated.

Moreover, iodine-containing contrast agents are preferred which are used in the imaging of vessels (angiography or phlebography) and in computer tomography (CT).

As iodine-containing contrast agents the following examples can be mentioned:

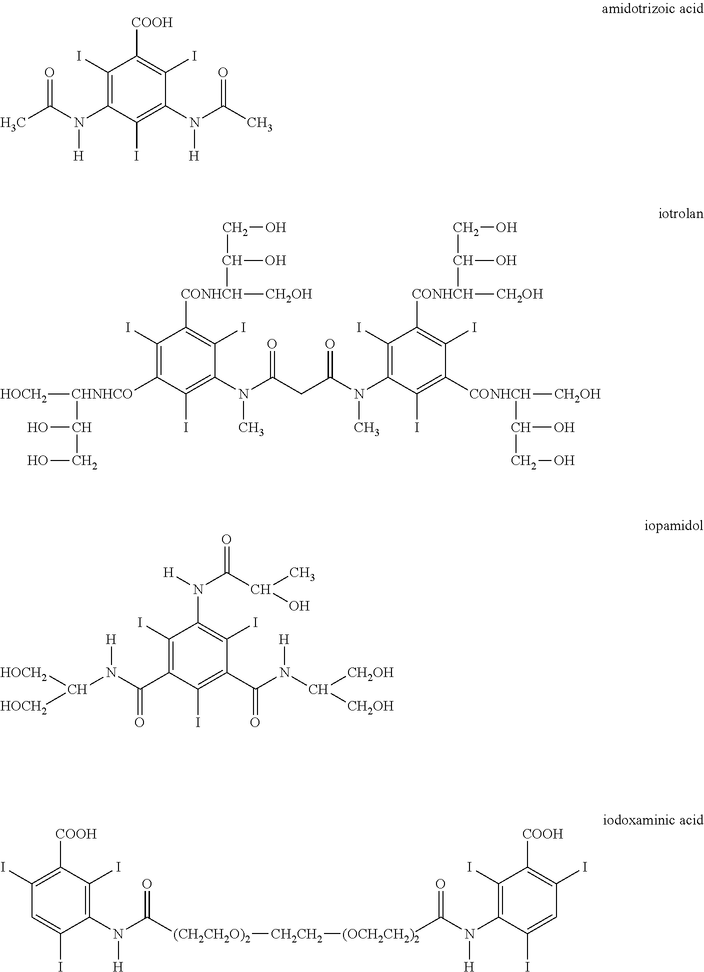

Another example is Jod-Lipiodol®, a iodinated Oleum papaveris, a poppy seed oil. Under the trademark Gastrografin® and Gastrolux® the mother substance of iodinated contrast agents, the amidotrizoate is commercially available in the form of sodium and Meglumin salts.

Also gadolinium-containing or superparamagnetic iron oxide particles as well as ferrimagnetic or ferromagnetic iron particles such as nanoparticles are preferred.

Another class of preferred contrast agents is represented by the paramagnetic contrast agents which contain mostly a lanthanide.

One of the paramagnetic substances which have unpaired electrons is e.g. gadolinium (Gd³⁺) which has in total seven unpaired electrons. Further in this group are europium (Eu²⁺, Eu³⁺), dysprosium (Dy³⁺) and holmium (Ho³⁺). These lanthanides can be used also in chelated form by using for example hemoglobin, chlorophyll, polyaza acids, polycarboxylic acids and especially EDTA, DTPA as well as DOTA as chelator.

Examples of gadolinium-containing contrast agents are gadolinium diethylenetriaminepentaacetic acid or

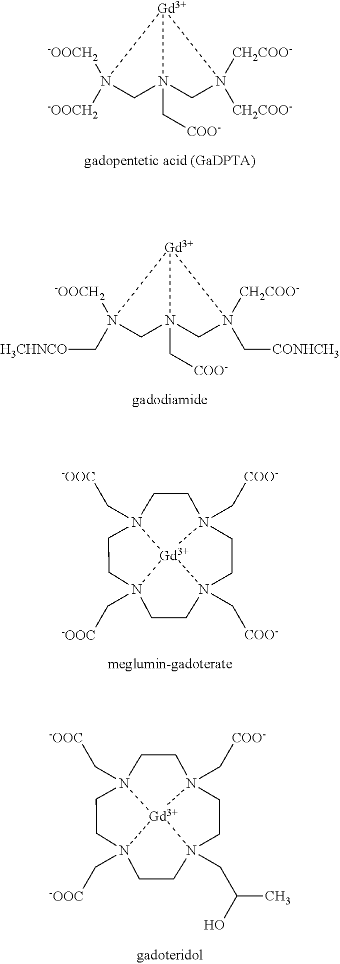

Further paramagnetic substances which can be used according to the invention are ions of so called transition metals such as copper (Cu²⁺), nickel (Ni²⁺), chromium (Cr²⁺, Cr³⁺), manganese (Mn²⁺, Mn³⁺) and iron (Fe²⁺, Fe³⁺). Also these ions can be used in chelated form.

The at least one substance which due to its ability of being excited by penetrating radiation allows for the detection of the basic body in in-vivo events by imaging methods is either on the surface of the basic body or inside the basic body.

In one preferred embodiment the balloon of the catheter is filled in its compressed form in the inside with a contrast agent and/or contrast agent analogue. The contrast agent is preferably present as a solution. Besides the properties of the contrast agent or contrast agent analogue as carrier or matrix for rapamycin such coatings have additionally the advantage that the catheter balloon is better visible, i.e. detectable, in the imaging methods. The expansion of the balloon takes place by expanding the balloon through further filling it with a contrast agent solution.

An advantage of this embodiment is that the contrast agent or contrast agent analogue can be reused any times and does not penetrate into the body and thus does not result in hazardous side effects.

As contrast agent analogues contrast agent-like compounds are referred to which have the properties of contrast agents, i.e. can be made visible with imaging methods that can be used during surgery.

Thus, further preferred embodiments of the present invention comprise catheter balloons coated with rapamycin and a contrast agent or a contrast agent analogue. If a coated or uncoated stent is present on the catheter balloon, of course, the balloon can be coated together with the stent. To this purpose rapamycin, optionally together with one or more other active agents, is dissolved or suspended in the contrast agent and applied to the catheter balloon with or without a stent. Moreover, the possibility exists to admix to the mixture of contrast agent and rapamycin a solvent which evaporates after coating or which can be removed under vacuum. Moreover, the possibility exists that under and/or on the contrast agent-containing layer additionally one or more layers of pure active agent or a polymer or an active agent-containing polymer is/are applied.

An especially preferred embodiment uses a catheter balloon with a crimped stent. The stent can be an uncoated (bare) stent or preferably a stent which is coated with only one hemocompatible layer. As hemocompatible coating are especially the heparin derivatives or chitosan derivatives preferred which are disclosed herein and especially desulfated and reacetylated or repropionylated heparin. The system of catheter balloon and stent is sprayed with or dipped into a solution or suspension or dispersion of rapamycin together with e.g. paclitaxel or thalidomide in a contrast agent (see example 20).

It is also possible to use specially designed catheter balloon such as fold balloons (or wing balloons or wrinkle balloons or balloons with folds or with wrinkles). Such fold balloons form folds (or wrinkles or wings) in the compressed state of the balloon which can be filled with an active agent such as pure rapamycin or with a mixture of rapamycin and a solvent or a contrast agent or a mixture of rapamycin and an oil or a polymer in a suitable solvent. An optionally used solvent can be removed under reduced pressure and thereby the mixture present in the folds can be dried. When dilatating such a fold balloon which is normally used without a stent, the folds turn or protrude to the outside and thus release their content to the vessel wall.

Another preferred embodiment of the stent or catheter balloon is in the use of transport mediators which accelerate or support the introduction of the active agent(s) into the cell. Often, these substances have a supporting or synergistic effect. These are comprised of e.g. vasodilators which comprise endogeneous substances such as kinins, e.g. bradykinin, kallidin, histamine or NOS-synthase which releases from L-arginin the vasodilatatory NO. Substances of herbal origin such as the extract of gingko biloba, DMSO, xanthones, flavonoids, terpenoids, herbal and animal dyes, food colorants, NO-releasing substances such as pentaerythrytiltetranitrate (PETN), contrast agents and contrast agent analogues belong also to these adjuvants or as such can be synergistically used as active agent.

Further substances to be mentioned are 2-pyrrolidon, tributyl- and triethylcitrate and their acetylated derivatives, bibutylphthalate, benzoic acid benzylester, diethanolamine, diethylphthalate, isopropylmyristate and -palmitate, triacetin etc.

### Stent Materials

The common stents which can be coated by methods according to the invention can be made of conventional materials such as medical stainless steel, titanium, chromium, vanadium, tungsten, molybdenum, gold, nitinol, magnesium, zinc, alloys of the aforementioned metals, or can be composed of ceramic materials or biostable and/or biodegradable polymers. These materials are either self-expandable or balloon-expandable and biostable and/or biodegradable.

### Balloon Materials

The catheter balloon can be comprised of usual materials, especially polymers, as they are described more below and especially of polyamide such as PA 12, polyester, polyurethane, polyacrylates, polyethers etc.

As mentioned in the beginning, besides the selection of the multipotent active agent rapamycin further factors are important to achieve a medical device which is optimally antirestenotically effective in the long-term. The physical and chemical properties of rapamycin and the optionally added further active agent as well as their possible interactions, active agent concentration, active agent release, active agent combination, selected polymers and coating methods represent important parameters which have a direct influence on each other and therefore have to be exactly determined for each embodiment. By regulating these parameters the active agent or active agent combination can be absorbed by the adjacent cells of the vessel wall in sufficient or optimally effective amount over the total restenosis-endangered critical period of time.

The stents according to the invention are provided preferably with at least one layer which contains the active agent rapamycin or a preferred active agent combination with rapamycin and which covers the stent completely or incompletely and/or the stent according to the invention contains the active agent rapamycin and/or an active agent combination with rapamycin in the stent material itself.

Additionally, by means of the hemocompatible layer on the surface it can be guaranteed during as well as after the diffusion of the active agent into the environment that no immune reactions occur against the foreign body.

On the one hand, the layers can be comprised of pure active agent layers, wherein at least one of the layers contains rapamycin, and on the other hand, of active agent-free or active agent-containing polymer layers or combinations thereof.

As methods for manufacturing such a medical device the spraying method, dipping method, pipetting method, electro-spinning and/or laser technique can be utilized. Depending on the selected embodiment the best-suitable method is selected for the manufacture of the medical device, wherein also the combination of two or more methods can be used.

Further preferred is the adding of at least another active agent which is either present with rapamycin in one layer or which is applied in a separate layer. As further combination the use of e.g. acetylsalicylic acid (aspirin) is advantageous because besides the supporting antiphlogisitc effect aspirin has also antithrombotic properties.

In the combination with the hydrophobic paclitaxel the antiproliferative effect can be increased or prolonged in dependence of the embodiment because paclitaxel and rapamycin complement one another by their different bioavailability. For example, the hydrophilic rapamycin layer can be applied to a paclitaxel layer, wherein rapamycin targets more the early occurring inflammatory reactions and paclitaxel inhibits the proliferation of the SMCs in the long-term.

Another preferred embodiment is the use of suitable biocompatible materials as reservoir for rapamycin or an active agent combination with rapamycin on the stent.

For this, the coating of a stent body with at least one biostable and/or bioresorbable polymer layer which contains rapamycin and/or an active agent combination of rapamycin is provided. The rapamycin content of the polymer layer is between 1% to 60% by weight, preferred between 5% to 50% by weight, especially preferred between 10% to 40% by weight.

Surprisingly, it was found that the use of biodegradable polymers is advantageous because the degradation of the polymers occurs as so-called bulk-erosion. The chain degradation takes place up to a certain degree with a substantial maintenance of the polymer's properties. Only after undershooting a certain chain length the material looses its properties and becomes brittle. The degradation occurs in the form of small detaching chips which are completely metabolized by the organism within a very short time. It was found that this degradation process can be used for a targetedly controlled increase of the rapamycin elution which offers a substantial improvement of restenosis prophylaxis.

While the elution of an active agent is normally especially high in the first days after implantation to have, as already discussed, a better control of the sum of acute defense reactions of the organism (to the wound itself and to the foreign body) this curve flattens in the further course quite rapidly such that the eluted active agent amount is steadily reduced until finally the elution is stopped and the still remaining active agent eluted from the polymer in a non-detectable way. However, according to the injury degree or patient habitus after 2-4 weeks reactions are noticed which require an increased dosing of active agent to limit restenosis.

By means of the timely controlled initiating lost of the polymer properties and degradation of a biodegradable polymer with the same drug-eluting stent an increase of the active agent elution which is important for restenosis porphylaxis can be achieved again at a predetermined later moment (see FIG. 4).

For example, the hydrolytic degradation of PLGA can be adjusted according to the mixture ratio of PLA to PGA or in the combination with other suitable polymers such that the elution curve has a further increased elution of rapamycin after more than 2 weeks. Depending on the combination of both components to each other or to other suitable polymers the dosing, moment and duration of the late and after a further moment again increased active agent's availability (“late burst”) can be adjusted exactly (see FIG. 4).

Additionally, it is possible with the use of at least one two-layer system to targetedly increase and/or expand the dosing and controlled active agent elution. This can be achieved e.g. when a first layer which is applied to the stent (or the hemocompatibly coated stent) has a higher concentration of rapamycin than the second polymer layer or a pure rapamycin layer which are applied to this first layer. The use of rapamycin-supporting active agents in the rapamycin-containing layer or in a layer which is existent separately from this layer is also possible.

Another preferred variant to increase the load of a drug-eluting stent with rapamycin is the inclusion of rapamycin in highly swellable substances such as alginate, pectine, hyaluronan, agar-agar, gum arabic, liposomal hydrogels, peptidehydrogels, gelatine capsules and/or highly swellable polymer such as PVP which are incorporated into the at least one biodegradable and/or biostable polymer layer. As further advantage the shielding of the active agent against the influences of the environment to the largest degree can be mentioned. Simultaneously, the possibility exists to add rapamycin and/or another active agent to the polymer layer which surrounds the active agent capsules.

With adding hydrophilic pore forming materials such as PVP besides the acceleration of the elution in the early phase of stent implantation also a more rapid degradation of the bioresorbable polymer is achieved due to the facilitated intrusion of water or plasma or cellular liquid into the polymer layer. In this way rapamycin is eluted more rapidly and in a higher dosage. This is of great advantage because the increased dosing positively affects the effectiveness, however, contrary to paclitaxel without resulting in necrotic alterations.

A special embodiment is the use of a biostable polymer as matrix and hydrophilic active agent-loaded materials (hydrophilic polymers such as PVP and/or micro-capsules and micro-beads from e.g. gelatine, alginate, cross-linked dextrins, gum arabic, agar-agar, etc.) as pore and/or channel forming materials. With adding aqueous media or implanting and expanding a suchlike coated stent the hydrophilic material will swell. As the swellability of the biostable polymer is low in comparison to the hydrophilic portion a pressure is generated in the pores due to the intrusion of liquid and the subsequent swelling such that the hydrophilic rapamycin is pressed out of the pores and channels like an injection into the vascular environment (see FIG. 5).

To increase the absorption of rapamycin into the cell's inside substances such as DMSO, lecithin and others of the mentioned transfection reagents can be added which increase the permeability of the cell membrane. This system can also be realized with biodegradable polymers as matrix. Decisive for this embodiment is the difference in the swellability of the substances used. Rapamycin is eluted to the extent to which the swellable material absorbs a liquid. Thus, the release of the active agent can be controlled by the rate of the liquid absorption. This system can also be realized with biodegradable polymers as matrix. Especially decisive is the difference in the swellability of the substances used.

Another embodiment which uses biostable polymers, especially polysulfones or polymerizable oils, can be provided such that in the polymeric surface of a biostably polymer coated stent holes are formed in a defined sequence by means of laser technology in which a rapamycin solution with or without added biodegradable polymer is incorporated by dipping or pipetting technology. A degradable polymer can be applied in this case as diffusion barrier either over the individual holes or on the total stent surface. In the event of this embodiment the vascular site of the stent can be treated in a targeted way. The adding of e.g. antithrombotics to the biostable polymers that cover also the inner side of the stent helps to minimize the risk of thrombosis which exists also on the luminal side.

According to this two-layer embodiment the first biostable layer is of a layer which is substantially covered by another biodegradable layer such that the above mentioned advantages of the active agent elution are maintained. Moreover, it is preferred to apply two polymer layers which consist either of the same or different materials, wherein rapamycin is present in one or in both layers in the same or in different concentration with or without further active agents.

The elution of rapamycin and/or an active agent combination can be controlled by adding pore forming agents such that in the two layers different amounts of pore forming agent are present, as well as by the possibility to targetedly incorporate different active agents which differently elute depending on the pore forming agent and its amounts in the coating.

After this two-layer embodiment the possibility exists to incorporate different active agents separately from each other into the layer which is suitable for the respective active agent such that e.g. a hydrophobic active agent is present in the one more hydrophilic layer and has another elution kinetics than another hydrophobic active agent which is present in the more hydrophobic polymer layer, or vice versa. This offers a broad field of possibilities to set the availability of the active agents in a certain reasonable sequence as well as to control the elution time and concentration.

As further preferred suitable polymers e.g. polycaprolactone, polycaprolactam, polyamino acids, trimethylenecarbonate and low-cross-linked polymerizable oils can be mentioned.

### EXAMPLES

### Example 1

### Spray Coating of Stents with Rapamycin

Purified, not expanded stents are horizontally hung onto a thin metal bar (d=0.2 mm), which is stuck on the rotation axis of the rotation and feed equipment and rotates with 28 r/min. The stents are fixed in that way, that the inside of the stents does not touch the bar and are sprayed with a 2% spray solution of rapamycin in chloroform or ethylacetate. Then, they are dried in the fume hood over night. If required, the coating process can be repeated until the desired active agent load is present on the stent.

### Example 2

### Determination of the Elution Behavior in PBS-Buffer

Per stent in a sufficient small flask 2 ml PBS-buffer is added, sealed and incubated in the drying closet at 37° C. After expiry of the chosen time intervals in each case the excess solution is depipetted and its UV absorption is measured.

### Example 3A

### Stent with Rapamycin as Basecoat and PVA as Topcoat

The rapamycin-spray coated and dried stent is spray coated in a second step with a methanolic-aqueous 1.5% PVA solution. Then, it is dried.

### Example 3B

### Spray Coating of Stents with Rapamycin and Cyclosporin A

Purified, not expanded stents are horizontally hung onto a thin metal bar which is stuck on the rotation axis of the rotation and feed equipment and rotates with 28 r/min. The stents are fixed in that way, that the inside of the stents does not touch the bar and are sprayed with a 2% spray solution of rapamycin and cyclosporin A in the ratio 2:0.5 in chloroform. Then, they are dried over night.

### Example 4

### Spray Coating of Stents with Rapamycin and Paclitaxel in Two Layers

Purified, not expanded stents are sprayed with a 0.8% spay solution of paclitaxel in chloroform. Then, the stent is dried at room temperature. In a second spaying process the method of example 1 is used.

### Example 5

### Coating of Stents with a Biodegradable or Biostable Polymer and Rapamycin or an Active Agent Combination with Rapamycin

Spray solution: 145.2 mg PLGA or polysulfone and 48.4 mg rapamycin or a 33% spray solution of a corresponding active agent combination of rapamycin (amount 20%-90%) with one or more other active agents such as paclitaxel, cyclosporin A, thalidomid, fusadil etc. are filled up with chloroform to 22 g. This spray solution is applied to the stent as already described.

The utilized stent can be a bare stent, a hemocompatibly coated stent and/or a stent coated with an active agent layer by spraying or dipping method. The pure active agent layer or active agent combination according to example 1 and 3 can be applied optionally on the polymer layer.

### Example 6

### Two-Layer System with a Biodegradable Polymer and Rapamycin or an Active Agent Combination with Rapamycin Having a Different Concentration of the Active Agent in the Layers

Solution 1: 25% solution of rapamycin or in combination with one or more active agents and PLGA in chloroform or optionally ethylacetate (0.8% solution)

Solution 2: 35% solution of rapamycin or in combination with one or more active agents and PLGA in chloroform or optionally ethylacetate (0.8% solution)

The stent is either a bare stent or a hemocompatibly coated stent and can have already a pure active agent layer of rapamycin, a combination with other active agents or a rapamycin-free active agent layer by dipping or spraying. Also, a pure active agent layer between the two polymer layers and/or as topcoat can be applied in a spraying or dipping method.

### Example 7

### Two-Layer System with a Biostable Polymer as Basecoat and a Biodegradable Polymer as Topcoat and Rapamycin or an Active Agent Combination with Rapamycin

PS-solution: 176 mg polyethersulfone are weighed in and filled up with chloroform to 20 g (0.88% solution)

PLGA-solution: 35% solution of rapamycin or in combination with one or more active agents (rapamycin content at least 20%) and PLGA (0.8% solution)

Also here, a bare stent or a hemocompatibly coated stent is used. After drying the first layer the biodegradable polymer layer can be applied, wherein the spraying and pipetting method which allow for a targeted application to the vascular stent are preferred. Also here, the active agent can be additionally applied between the two polymer layers and/or on the surface as additional layer by spraying, dipping or pipetting method.

### Example 8

### Coating of a Stent with Biostable or Biodegradable Polymer Having a High Content of a Hydrogel (PVP, Silicon, Hydrosome, Alginate, Peptide, Glycosaminoglycane) as Pore Forming Agent (or Channel Forming Agent)

Rapamycin (or an active agent combination, 35% by weight) is dissolved with polysulfone and hydrogel in chloroform such that a solution is formed which contains 8% hydrogel. This solution is applied to the stent as in the above examples. The total concentration of the polymer solution should be below 0.9% to achieve an optimal spraying behavior. In the dipping method the solution should not have above 30% polymer content. The rapamycin loading can also be done by subsequent dipping of the already coated stent into an active agent solution (2%).

### Example 8a

### Spray Solution Polysulfone/PVP without Addition of Rapamycin

24 mg PS and 2.4 mg PVP are weighed in and filled up with chloroform to 3 g
→0.80% PS, 0.08% PVP

### Example 8b

### Spray Solution Polysulfone/PVP with Addition of Rapamycin

18.2 mg PS, 14.1 mg rapamycin and 3.2 mg PVP are weighed in and filled up with chloroform to 4 g
→0.45% PS, 0.35% Rapamycin, 0.08% PVP

### Example 9

### Covalent Hemocompatible Coating of Stents

### a) Preparation of Desulfated Reacetylated Heparin:

100 ml of amberlite IR-122 cation exchange resin were filled into a column having a diameter of 2 cm, transformed into the H⁺ form with 400 ml 3M HCl and washed with distilled water until the eluate was free from chloride and pH neutral. 1 g of sodium heparin was dissolved in 10 ml of water, put onto the cation-exchange column and eluted with 400 ml of water. The eluate was allowed to drop into a receiver with 0.7 g of pyridine and subsequently titrated with pyridine to pH 6 and freeze-dried.

0.9 g of heparin pyridinium salt were added to 90 ml of a 6/3/1 mixture of DMSO/1,4-dioxane/methanol (v/v/v) in a round bottomed flask with reflux cooler and heated to 90° C. for 24 hours. Then, 823 mg of pyridinium chloride were added and heating to 90° C. was effected for further 70 hours. Subsequently, dilution was carried out with 100 ml of water, and titration to pH 9 with dilute soda lye was effected. The desulfated heparin was dialyzed against water and freeze-dried.

100 mg of the desulfated heparin were dissolved in 10 ml of water, cooled to 0° C. and mixed with 1.5 ml of methanol under stirring. To the solution, 4 ml of Dowex 1×4 anion-exchange resin in the OH⁻ form and subsequently 150 μl of acetic acid anhydride were added and stirred for 2 hours at 4° C. After that, the resin is filtrated, and the solution is dialyzed against water and freeze-dried.

### b) N-Carboxymethylated, Partially N-Acetylated Chitosan:

In 150 ml 0.1 N HCl, 2 g of chitosan were dissolved and boiled under nitrogen for 24 hours under reflux. After cooling to room temperature, the pH of the solution was adjusted to 5.8 with 2 N NaOH. The solution was dialyzed against demineralized water and freeze-dried.

1 g of the chitosan partially hydrolyzed this way was dissolved in 100 ml of a 1% acetic acid. After adding 100 ml of methanol, 605 μl of acetic acid anhydride dissolved in 30 ml of methanol were added and stirred for 40 minutes at room temperature. The product was precipitated by pouring into a mixture of 140 ml of methanol and 60 ml of a 25% NH₃ solution. It was filtrated, washed with methanol and diethyl ether and dried under vacuum over night.

1 g of the partially hydrolyzed and partially N-acetylated chitosan was suspended in 50 ml of water. After adding 0.57 g of glyoxylic acid monohydrate, the chitosan derivative dissolved within the next 45 minutes. The pH value of the solution was adjusted to 12 with 2 N NaOH. A solution of 0.4 g of sodium cyanoboron hydride in as few water as possible was added and stirred for 45 minutes. The product was precipitated in 400 ml of ethanol, filtrated, washed with ethanol and dried in vacuum over night.

### c) Preparation of Desulfated N-Propionylated Heparin:

100 ml of amberlite IR-122 cation exchange resin were filled into a column having a diameter of 2 cm, transformed into the H⁺ form with 400 ml 3M HCl and washed with distilled water until the eluate was free from chloride and pH neutral. 1 g of sodium heparin was dissolved in 10 ml of water, put onto the cation-exchange column and eluted with 400 ml of water. The eluate was allowed to drop into a receiver with 0.7 g of pyridine and subsequently titrated with pyridine to pH 6 and freeze-dried.

0.9 g of heparin pyridinium salt were added to 90 ml of a 6/3/1 mixture of DMSO/1,4-dioxane/methanol (v/v/v) in a round bottomed flask with reflux cooler and heated to 90° C. for 24 hours. Then, 823 mg of pyridinium chloride were added and heating to 90° C. was effected for further 70 hours. Subsequently, dilution was carried out with 100 ml of water, and titration to pH 9 with dilute soda lye was effected. The desulfated heparin was dialyzed against water and freeze-dried.

100 mg of the desulfated heparin were dissolved in 10 ml of water, cooled to 0° C. and mixed with 1.5 ml of methanol under stirring. To the solution, 4 ml of Dowex 1×4 anion-exchange resin in the OH⁻ form and subsequently 192 μl of propionic acid anhydride were added and stirred for 2 hours at 4° C. After that, the resin is filtrated, and the solution is dialyzed against water and freeze-dried.

### d) N-Carboxymethylated, Partially N-Propionylated Chitosan:

In 150 ml 0.1 N HCl, 2 g of chitosan were dissolved and boiled under nitrogen for 24 hours under reflux. After cooling to room temperature, the pH of the solution was adjusted to 5.8 with 2 N NaOH. The solution was dialyzed against demineralized water and freeze-dried.

1 g of the chitosan partially hydrolyzed this way was dissolved in 100 ml of a 1% acetic acid. After adding 100 ml of methanol, 772 μl of propionic acid anhydride dissolved in 30 ml of methanol were added and stirred for 40 minutes at room temperature. The product was precipitated by pouring into a mixture of 140 ml of methanol and 60 ml of a 25% NH₃ solution. It was filtrated, washed with methanol and diethyl ether and dried under vacuum over night.

1 g of the partially hydrolyzed and partially N-acetylated chitosan was suspended in 50 ml of water. After adding 0.57 g of glyoxylic acid monohydrate, the chitosan derivative dissolved within the next 45 minutes. The pH value of the solution was adjusted to 12 with 2 N NaOH. A solution of 0.4 g of sodium cyanoboron hydride in as few water as possible was added stirred for 45 minutes. The product was precipitated in 400 ml of ethanol, filtrated, washed with ethanol and dried in vacuum over night.

### Example 10

### Covalent Hemocompatible Coating of Stents

Non-expanded stents made of medical stainless steel LVM 316 were degreased in the ultrasonic bath for 15 minutes with acetone and ethanol and dried at 100° C. in the drying oven. Subsequently, they were dipped into a 2% solution of 3-aminopropyltriethoxysilane in an ethanol/water mixture (50:50: (v/v)) for 5 minutes and dried at 100° C. Subsequently the stents were washed with dematerialized water.

3 mg of the hemocompatible substance of example 10 (e.g. desulfated and reacetylated heparin) was dissolved at 4° C. in 30 ml of 0.1 M MES buffer (2-(N-morpholino)ethanesulfonic acid) pH 4.75 and mixed with 30 mg of N-Cyclohexyl-N′-(2-morpholinoethyl)carbodiimide-methyl-p-toluenesulfonate. 10 stents were stirred at 4° C. during 15 hours in this solution. Subsequently, they were rinsed with water, 4 M NaCl solution and water for respectively 2 hours.

### Example 11

### Determination of the Glucosamine Content of the Coated Stents by HPLC

Hydrolysis: The coated stents were transferred into small hydrolysis tubes and left with 3 ml 3 M HCl for exactly one minute at room temperature. The metal samples were removed and after sealing the tubes were incubated for 16 h at 100° C. in the drying oven. Then, they were allowed to cool down, it was evaporated three times until dryness and transferred into 1 ml degassed and filtered water and measured against an also hydrolyzed standard in the HPLC.

| Stent | Surface | Ac-heparin | Surface | Ac-heparin | Ac-heparin |
|---|---|---|---|---|---|
| No. | sample | [g/sample] | [cm²] | [g/cm²] | [pmol/cm²] |
| 1 | 129.021 | 2.70647E−07 | 0.74 | 3.65739E−07 | 41.92 |
| 2 | 125.615 | 2.63502E−07 | 0.74 | 3.56084E−07 | 40.82 |
| 3 | 98.244 | 1.93072E−07 | 0.74 | 2.60908E−07 | 29.91 |
| 4 | 105.455 | 2.07243E−07 | 0.74 | 2.80058E−07 | 32.10 |
| 5 | 119.061 | 2.33982E−07 | 0.74 | 3.16192E−07 | 36.24 |
| 6 | 129.202 | 2.53911E−07 | 0.74 | 3.43124E−07 | 39.33 |
| 7 | 125.766 | 2.53957E−07 | 0.74 | 3.43185E−07 | 39.34 |

### Example 12

### Biocompatible Coating of Stents with Linseed Oil Under Addition of a Catalyst and a Synthetic Polymer, Especially Polyvinylpyrrolidone and Subsequent Addition of Active Agent

a) Non expanded stents of medical stainless steel LVM 316 are removed from fat in the ultrasonic bath for 15 minutes with acetone and ethanol and dried at 100° C. in the drying oven. Subsequently the stents are washed with demineralized water over night.

About 10 mg of KMnO, are dissolved in 500 μl of water and as much as possible PVP is added. The mixture is spread laminarly on a polypropylene substrate and allowed to dry at room temperature over night.

From this brittle mixture 2.5 mg are dissolved in 1 ml of chloroform and the resulting solution is sprayed after adding of 10.5 μl of linseed oil with an airbrush spraying pistol (EVOLUTION from Harder & Steenbeck) from a distance of 6 cm on a rotating 18 mm LVM stainless steel stent. Afterwards the coated stent was stored for 24 h at 80° C.

b) Addition of active agent to a coated stent in the dipping method

The coated stent of example 18a) was dipped into a solution of 800 μg of rapamycin in 1 ml of ethanol and allowed to swell. After accomplishing the swelling process the stent was extracted and dried.

### Example 13

### Biocompatible Coating of Stents with Linseed Oil and Rapamycin

Non expanded stents of medical stainless steel LVM 316 are removed from fat in the ultrasonic bath for 15 minutes with acetone and ethanol and dried at 100° C. in the drying oven. Subsequently the stents were washed with demineralized water over night.

Linseed oil and rapamycin (70:30) are dissolved in the mixture ratio of 1:1 in chloroform and then sprayed on the continuously rotating stent. After evaporation of the chloroform in the soft air stream the stent is stored in the drying oven at 80° C. The average coating mass is 0.15 mg±0.02 mg.

### Example 14

### Biocompatible Coating of Rapamycin-Eluting Stents with an Ethanol Spraying Solution Of Linseed Oil and the Synthetic Polymer Polyvinylpyrrolidone (PVP)

After cleaning the stents as already described in the examples before an ethanol spraying solution is prepared which contains 0.25% linseed oil and 0.1% PVP and continuously sprayed with a spraying pistol on the stent rotating around its axis. Then it is dried over night at 70° C. The average coating mass is 0.2 mg±0.02 mg.

Rapamycin or an active agent combination with rapamycin is either incorporated subsequently by swelling or admixed to the spraying solution with at least 20% by weight of rapamycin content.

### Example 15

### Biocompatible Coating of Stents with Linseed Oil and the Synthetic Polymer Polyvinylpyrrolidone (PVP) in the Two-Layer System with Addition of a Restenosis-Inhibiting Active Agent

After cleaning of the stents a first layer of 0.35% by weight of rapamycin dissolved in chloroform is sprayed on the stent. After drying of this layer at room temperature the second layer of a chloroform solution with 0.25% linseed oil and 0.1% PVP is sprayed on.

### Example 16

### Biocompatible Coating of Stents with Linseed Oil and α-Linolenic Acid

After cleaning the stents with acetone and ethanol as previously described a mixture solved in ethanol with 0.20% linseed oil and 0.5% a-linolenic acid is prepared and continuously sprayed on the stent.

### Example 17

### Complete Coating of an Esophagial Stent by Dip-Coating

### a) Precoating of Stent Struts

A stent is fixed on the rod of a rotator and is sprayed with 1% polyurethane solution at very slow rotational speed by slowly moving the pistol upwards and downwards. After being sprayed, the stent is of a mat gray color, such that an optical spray control can be conducted. It is particularly important that the edge is sprayed accurately which can be ensured by additional circumferential spraying. Subsequently, the stent is allowed to dry.

### b) Complete Coating of a Stent Sprayed According to a)

Polyurethane and 35% by weight of rapamycin/terguride (4:1) are dissolved in THF, so that a 14% solution is obtained. A stent precoated according to example 18a) is carefully mounted on the adequate mold. The tool with the stent mounted thereon is immersed head first into pure THF until rising air bubbles can be seen. Subsequently, the stent is slowly immersed into the 14% polyurethane solution. After 15 seconds, the core is slowly removed and immediately oriented horizontally and the core is turned so that the PU is uniformly distributed on the stent and allowed to dry.

Once the PU has stopped running, the core is allowed to dry under the fume hood and subsequently tempered at 95° C. during 45 min in the drying oven. After cooling it is dipped into a warm 0.3% SDS solution for detaching the stent from the tool. After purification under running water and rinsing with 0.5 m NaOH, it is thoroughly rinsed under running water and in DI water.

### Example 18

### Partial Coating of a Neuronal Stent for the Treatment of Aneurysms

Solution: 3.2 mg of PU dissolved in 20 ml of N-methyl-2-pyrrolidone and 33% by weight of rapamycin

A spray-coated stent is pushed on an adequate, freely rotatable mold such that it completely contacts the smooth surface. The application of the coating is done in at least two steps, wherein solution is taken with a brush hair which is applied on the field to be coated until the field is completely covered with solution. If each of the selected fields to be coated is filled with the desired coating thickness the stent is dried at 90° C. After cooling down the stent is detached from the mold.

### Example 19

### Coating of a Fold Balloon with Rapamycin by Means of Spraying Method

After careful prewetting of the balloon with acetone the balloon is continuously sprayed with a solution of rapamycin in ethylacetate during rotation around the longitudinal axis and dried. For preventing the folds (or wrinkles or wings) from defolding during rotation the balloon is set under vacuum.

### Example 20

### Complete Coating of a Fold Balloon with Rapamycin by Means of Pipetting Method

The fold balloon is fixed in horizontal position to a rotatable axis. For preventing the folds from defolding during rotation the balloon is set under vacuum. Thus, step by step the ethanol-dissolved active agent is applied along the longitudinal axis at the outside and inside the folds with a teflon canula as extension of a syringe tip until a continuous rapamycin layer can be observed. Then the balloon is dried.

Preferably an adjuvant which facilitates the permeability of the active agent into the cells is added to the active agent solution. For example, 150 mg of rapamycin, 4.5 ml of acetone, 100 μl of iodopromide and 450 μl of ethanol are mixed.

### Example 21

### Determination of the Active Agent Losses by Expansion in an In-Vitro Model

The fold balloon coated with rapamycin and an adjuvant is expanded in a silicon hose which is filled with PBS buffer. Then the remaining coating on the balloon is dissolved in a defined amount of acetonitrile and the rapamycin content is quantified by HPLC. Moreover, the amount of rapamycin which adheres at the wall of the hose is purged with acetonitrile and quantified, the amount in the buffer is also determined.

### Example 22

### Partial Coating of a Fold Balloon (or Wing Balloon) with Rapamycin by Means of Pipetting Method

The fold balloon (or wing balloon) is fixed in horizontal position on a rotatable axis such that the fold to be filled is always on the top side and vacuum is applied for preventing the fold from opening. A 1% low-viscous alcoholic solution of rapamycin is prepared which is such low-viscous that the solution can soak itself into the folds of a fold balloon (or wing balloon) due to capillary forces. By means of a capillary which contacts an end of the fold the alcoholic solution is allowed to flow into the fold until the inside of the fold is completely filled due to capillary forces. The content of the fold is allowed to dry, the balloon is turned and the next fold is filled. Each fold (or wrinkle) is filled only once.

### Example 23

The balloon of example 22 which is loaded with active agent only in the folds can be coated in a second step by spraying method with a polymeric external layer as barrier. The concentration of the polymer spray solution has to be kept as low as possible such that the polymer layer resulting after drying does not interfere with the continuous opening. For example, already a 0.5% PVP-solution is suitable.

### Example 24

### Coating of an Inflated Catheter Balloon Exclusively in the Folds in the Presence of a Stent Crimped on the Balloon

a) A 35% solution of rapamycin or an active agent combination (e.g. rapamycin and thalidomide or thalidomide/paclitaxel mixture) in chloroform is applied to the folds of a fold balloon (or wing balloon) which is rotatably mounted by a pipetting device until it is visible that the folds are continuously filled. Then the fold balloon is dried under slow rotation at room temperature. The presence of a stent or drug-eluting stent crimped on the balloon does not interfere with the process.

b) A biostable or biodegradable polymer or a combination of both (see the previous examples) and an active agent combination with at least 30% by weight of rapamycin are dissolved with chloroform such that the total active agent amount of the solution is 30% by weight. The total solution is 0.9%. This solution can also be applied according to the dipping or spraying methods. Also here, the stent can be present already.

1-9. (canceled)

10. Stent coated with at least one polymer layer of poly(lactic-co-glycolic acid), polylactic acid, polyglycolic acid, polyvinylpyrrolidone, polypropylene glycol, polyethylene glycol or polyvinyl alcohol and derivatives or combinations of these polymers and rapamycin.

11. Stent according to claim 10, wherein the rapamycin on the stent surface is covered by the polymer layer, wherein the polymer layer allows rapamycin to be continuously eluted through the polymer layer in at least two increasing steps within the elution graph, which are time- and dosis-adapted to the needs of restenosis prophylaxis.

12. Stent according to claim 11, wherein the elution graph of rapamycin has two inclinations.

13. Stent according to claim 10 coated with a polymer layer of PLGA which contains rapamycin.

14. Stent according to claim 10, wherein the stent has a lowermost coating of a hemocompatible polymer.

15. Stent according to claim 14, wherein the hemocompatible coating is bound covalently to the stent surface.

16. Stent according to claim 10, wherein rapamycin is present in combination with a second active agent.

17. Stent according to claim 16, wherein the second active agent is selected from the group comprising: tacrolimus, paclitaxel and its derivatives, Fasudil®, vitronektin receptor antagonists, thalidomid, cyclosporin A, tergurid, lisurid, celecoxip.

18. Stent according to claim 10, wherein rapamycin has a concentration from 0.001-10 mg per cm2 of stent surface.

19. Stent according to claim 10, wherein not only the stent struts but also the interstices between the stent struts are coated with the polymer in which rapamycin is present.

20. Stent according to claim 10, wherein the stent is formed by a continuous or sequential spraying method, a dipping method, a brushing method, a pipetting method, electro-spinning, a soap bubble method, a plasma method and/or a laser technique.
